(19) United States
(12) Patent Application Publication
(10) Pub. No.: US 2025/0146088 A1
(43) Pub. Date: May 8, 2025

(54) COMPOSITIONS AND METHODS OF GENERATING A SIGNAL FROM ONE OR MORE PROTEIN-OLIGONUCLEOTIDE REPORTERS

(71) Applicant: The Research Foundation for the State University of New York, Albany, NY (US)

(72) Inventor: Brian Patrick Callahan, Vestal, NY (US)

(21) Appl. No.: 18/212,537

(22) Filed: Jun. 21, 2023

Related U.S. Application Data
(60) Provisional application No. 63/355,288, filed on Jun. 24, 2022.

(51) Int. Cl. C12Q 1/70; C12Q 1/6818

(52) U.S. Cl. CPC C12Q 1/701; C12Q 1/6818

(57) ABSTRACT

The present disclosure relates to kits, compositions, or one or more methods of detecting an analyte-of-interest using one or more protein-oligonucleotide reporters of the present disclosure. For example, the present disclosure relates to a composition, including: a conjugate reaction product of: a signal emitting peptide or functional fragment thereof having a predetermined electrophilic residue; a linker characterized as a fused sterol or stanol ring system, having a nucleophilic group at a 3-position of an A-ring of a fused sterol or stanol ring system with beta or alpha stereochemistry; and an oligonucleotide having a 5′ end and a 3′ end, wherein the composition is characterized by one of 3′ end or 5′ end attached to the linker and one of 3′ end or 5′ end attached to a quencher, and wherein the linker is covalently linked to the predetermined electrophilic residue.

### CROSS-REFERENCE TO RELATED APPLICATIONS

The present disclosure claims priority or the benefit under 35 U.S.C. § 119 of U.S. provisional application No. 63/355,288 filed Jun. 24, 2022, herein entirely incorporated by reference.

### STATEMENT OF GOVERNMENTAL SUPPORT

This invention was made with governmental support under grant nos. CA206592 and Al163907 awarded by The National Institutes of Health. The government has certain rights in this invention.

### REFERENCE TO A SEQUENCE LISTING

This application contains a Sequence Listing in computer readable form, which is incorporated herein by reference. File name 020-RB669US01, created on Jul. 3, 2024, size of the file is 13 kilobytes.

### FIELD OF THE INVENTION

The present disclosure is in the field of biotechnology and signal generation. In embodiments, a signal may indicate the presence of one or more analytes-of-interest using one or more protein-oligonucleotide reporters of the present disclosure. The disclosure also relates to binding one or more protein-oligonucleotide reporters of the present disclosure to a predetermined nucleic acid to change the shape of the one or more protein-oligonucleotide reporters from a first conformation to a second conformation, wherein the second conformation generates or bolsters a signal.

### BACKGROUND

Reagents for the specific detection of one or more analytes-of-interest such as dilute nucleic acid(s) are fundamental to molecular and cellular genetics and various clinical diagnostics, including tests for viral pathogens like SARS-COV-2. Fluorogenic hairpin-forming oligonucleotide-based sensors, such as molecular beacons, have provided a standard detection tool. Modified with fluorophore and quencher at opposite ends, the oligonucleotide fluorescence is suppressed in the hairpin, or off state. Molecular beacons switch on by hybridizing to complementary nucleic acid, which separates the fluorophore and quencher, increasing radiative emission. However, molecular beacons are deficient in that they may be difficult to use due to problematic low signal emission or strength from fluorophores, and/or lack of robust compositions for amplifying a light source. Accordingly, molecular testing is often stymied by false positives, false negatives, and time-consuming product analysis. Moreover, the fluorogenic compositions are deficient in that copious amounts of reagents are required for use thereof.

Accordingly, there is a continuous need for improved signal generating and/or amplification using one or more biomolecules in biochemical assays. Such compounds would be extremely useful as biosensors to enhance sensitivity, specificity and speed of isothermal amplification techniques such as nucleic acid sequence-based amplification (NASBA) and reverse transcription loop-mediated isothermal amplification (RT-LAMP).

### SUMMARY

The present disclosure now provides compounds such as biomolecules for signal generating and/or amplification in biochemical assays. Such compounds, or protein-oligonucleotide reporters of the present disclosure, are extremely useful as biosensors to enhance sensitivity, specificity and speed of isothermal amplification techniques such as nucleic acid sequence-based amplification (NASBA) and reverse transcription loop-mediated isothermal amplification (RT-LAMP).

Embodiments of the present disclosure include excellent biomolecules such as protein-oligonucleotide reporters of the present disclosure capable of generating a signal such as light. In embodiments, biocatalysis is employed in the preparation of a biosensor molecule such as a protein-oligonucleotide reporters of the present disclosure for the sensor output, and the molecules, in embodiments, include a compact, ATP-independent bioluminescent enzyme, nanoluciferase (Nluc) or a functional fragment thereof. This substitution provides an internal, amplifiable light source. Nluc with the engineered substrate such as coelenterazine, or furimazine produces light that is sufficiently bright for measurement using a portable luminometer. With detection signal enzymatically amplified, it has been found that nucleic acid detection assays consume less reagent while improving sensitivity relative to synthetic molecular beacons.

In embodiments, the present disclosure provides turn-on enzyme biosensors, or protein-oligonucleotide reporters, to serve as detection agents for analytes-of-interest by way of pathogen molecular testing and the like. Non-limiting examples of analytes-of-interest include nucleic acids from or derived from aberrant cellular nucleic acids, such as nucleic acids from a tumor, a cancer cell, or viral DNA, viral RNA, and/or SARS-COV-2. Further, the biosensors of the present disclosure such as one or more protein-oligonucleotide reporters of the present disclosure enhance sensitivity, specificity, and speed of isothermal amplification techniques, such as NASBA and RT-LAMP.

In embodiments, the present disclosure includes a composition, including: a conjugate reaction product of: a signal emitting peptide or functional fragment thereof having a predetermined electrophilic residue, i.e., a glycine amino acid residue located at the final residue position of the light emitting polypeptide; a linker characterized as a fused sterol or stanol ring system, having a nucleophilic group at a 3-position of an A-ring of a fused sterol or stanol ring system with beta or alpha stereochemistry; and an oligonucleotide having a 5′ end and a 3′ end, wherein the composition is characterized by one of the 3′ end or the 5′ end attached to the linker and one of the 3′ end or the 5′ end attached to a quencher, and wherein the linker is covalently linked to the predetermined electrophilic residue, i.e., a glycine amino acid residue.

In embodiments, the present disclosure includes a composition, including: a peptide reporter molecule, and a hairpin-forming mono-sterylated oligonucleotide including a quencher.

In embodiments, the present disclosure includes method of making a composition, including: contacting a light emitting polypeptide or functional fragment thereof having an predetermined electrophilic residue with a linker characterized as a fused sterol or stanol ring system having a nucleophilic group at a 3-position of an A-ring of a fused sterol or stanol ring system with beta or alpha stereochemistry, and an oligonucleotide having a 5′ end and a 3′ end or the 5′ end or the 3′ end attached to the quencher.

In embodiments, the present disclosure includes a method of determining a presence of an analyte-of-interest, including: contacting a composition including a light emitting polypeptide or functional fragment thereof, a linker characterized as a fused sterol or stanol ring system, and an oligonucleotide having a 5′ end attached to the linker and a 3′ end attached to a quencher with an analyte of interest under conditions sufficient to separate the light emitting polypeptide or functional fragment thereof from the quencher to generate a signal.

In embodiments, the present disclosure includes a method of reconfiguring a protein-nucleic acid fusion molecule, including contacting a protein-nucleic acid fusion molecule, having a first conformation, with an oligonucleotide-of-interest to form a protein-nucleic acid fusion molecule complex having a second conformation, wherein the second conformation is characterized as on; and contacting the second conformation with an indicator under conditions sufficient to form a signal.

In embodiments, the present disclosure includes a composition, including: an enzyme-nucleic acid molecule including one or more nucleic acid binding sites, wherein the enzyme-nucleic acid molecule has a first conformation characterized as off, and a second conformation characterized as on when in a presence of an analyte-of-interest.

The illustrative aspects of the present disclosure are designed to solve the problems herein described and/or other problems not discussed

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C depict a method of the present disclosure and biocatalytic preparation of a composition of the present disclosure. FIG. 1A depicts a scheme showing HhC catalyzed bioconjugation of Nluc (grey) to a hairpin-forming mono-sterylated oligonucleotide (steramer) modified with a 3′ dark quencher (Q). In embodiments, conjugation involves formation of an internal thioester (step 1), followed by steramer binding and acyl transfer to the sterol hydroxyl group (step 2). FIG. 1B depicts a general mechanism for turn-on nucleic acid detection using a composition of the present disclosure, where hybridization of the Nluc conjugated hairpin oligonucleotide with complementary nucleic acid displaces the quencher (Q) from Nluc, enhancing bioluminescence signal. FIG. 1C depicts a preparation of a composition of the present disclosure monitored by SDS-PAGE. A fusion of Nluc with HhC is purified first by Ni-NTA chromatography, followed by size exclusion chromatography (SEC), then reacted with steramer (conjugation). Composition of the present disclosure such as E-beacon (Eb.) is isolated by agarose gel extraction. Nucleic acid was visualized by UV with GelRed stain.

FIGS. 2A-2C depict a proof of concept for the composition of the present disclosure and sequence specific nucleic acid detection. FIG. 2A depicts bioluminescence from Eb. 1 increases in the presence of complementary oligonucleotide (green, n=48) compared to composition of the present disclosure or E-beacon samples mixed with noncomplementary oligonucleotide (red, n=48). FIG. 2B depicts results of “blinded test” of Eb.1 specificity indicate that Eb.1 distinguishes complementary oligonucleotide (green) from oligonucleotides containing 1-3 base mismatches. See also Table 1 for sequence information. FIG. 2C depicts a comparison of compositions of the present disclosure such as E-beacons with different quenchers: lowaBlack (Eb.1), dabcyl (Eb.2), and ONYX-A (Eb.3). Bioluminescence was measured after 10-minute incubation with complementary oligonucleotide (green, n=48) or noncomplementary oligonucleotide (red, n=48). * In FIGS. 2A, 2B, and 2C the composition of the present disclosure such as an E. beacon was present at 2×10⁻⁹ M final; oligonucleotide at 25×10⁻⁹ M; temperature, 25° C.; substrate, furimazine.

FIGS. 3A-3C depict compositions of the present disclosure such as E-beacons for SARS-COV-2. FIG. 3A depicts bioluminescence readings of samples with Eb.19 (WT) after 10 min. 1 hr or 3 hr incubation with increasing concentration of complementary target oligonucleotide. The curves show hyperbolic binding isotherms with calculated half maximum emission (EC₅₀) at 2.54×10⁻⁹ M of oligonucleotide using the 10 min incubation, 0.2×10⁻⁹ M for the 1 hr incubation and 0.11×10⁻⁹ M for the 3 hr incubation. FIG. 3B depicts a single base-pair mismatch discrimination by composition or E-beacon, Eb.19 (WT). Bioluminescence from Eb.19 (WT), at 80×10⁻¹² M, is plotted as a function of increasing oligonucleotide concentration. E484 is the complementary target, E484K is single base pair mismatch, “random” is non-complementary (see also Table 1). Samples were incubated for 3 hrs followed by bioluminescence measurement. FIG. 3C depicts bioluminescence spectrum measured by RLU/see showing the selective detection of E484K oligonucleotide by E-beacon, Eb.19 (E484K). Oligonucleotides E484 and E484Q represent single base pair variants of wild type SARS-COV-2 and Kappa SARS-COV-2, respectively.

FIGS. 4A and 4B depict composition of the present disclosure such as E-beacon (Eb.2′) prepared with solid phase synthesized mono-sterylated oligo displays favorable signal to noise ratio and limited data scatter. FIG. 4A depicts steramer and test oligo sequences. Signal: green; Noise: red. FIG. 4B depicts bioluminescence from samples with Eb.2′ after 10-minute incubation with complementary oligonucleotide (signal, green, n=48) or with noncomplementary oligonucleotide (noise, red, n=48). The E-beacon was present at 8×10⁻¹¹ M final; oligonucleotide at 1×10⁻⁷ M; temperature, 25° C.; substrate, furimazine.

FIG. 5 depicts S/N ratio exceeds 5-fold (above the red dotted line) with sub-picomolar Eb.19 (WT). Target oligonucleotides was held constant at 1×10⁻⁷ M while the Eb. 19 (WT) was titrated from 1×10⁻⁸ M to 1×10⁻¹⁴ M. Signal to noise ratios are plotted on the Y axis.

FIG. 6 depicts bioluminescence of Eb. 19 (E484K) with increasing target oligo concentration after 10 min, 1 hr, 3 hr incubation. Data was fit to a hyperbolic binding isotherm to calculate EC₅₀ values. The EC₅₀ was 2.54×10⁻⁹ M for 10 min incubation; for 1 hr, EC₅₀ was 0.62×10⁻⁹ M; for 3 hr, EC₅₀ was 0.76×10⁻⁹ M. Eb. 19 (E484K) was present at 8×10⁻¹¹ M.

FIGS. 7A and 7B depict unquenching of Eb.2′ by digestion of the hairpin component of Eb.2′ with DNASE-1 produced similar S/N ratios compared to Eb.2′ probe hybridization driven unquenching. FIG. 7A depicts digestion of steramer separates Nluc and quencher. FIG. 7B depicts Eb.2′ (1×10⁻⁸ M, final) unquenching after mixing with DNASE-1 (6 units) in DNASE-1 buffer (from New England Biolabs) at 25° C. and analyzed for bioluminescence at selected intervals. The S/N was calculated from the ratio of bioluminescence in samples +/− DNASE-1. The incubation period varies from 0 min to 90 min.

FIG. 8 depicts Eb. 19 (WT) is sufficiently thermostable and maintains robust signal/noise after 30 min incubation at 65° C., typical for Loop-Mediated Isothermal Amplification (LAMP). Longer incubations diminish S/N ratio. Samples at room temperature are included as controls. Eb.19 (WT) (1×10⁻⁸ M, final) with complementary or noncomplementary oligonucleotide (1×10⁻⁷ M, final).

FIG. 9 depicts the overall bioluminescent signal of Eb. 19 (WT) (1×10⁻⁸ M, final) diminishes after extended incubation at 65° C., typical of Loop-Mediated Isothermal Amplification (LAMP). Complementary (target) and noncomplementary (random) oligonucleotide added at 1×10⁻⁷ M, final.

FIGS. 10A and 10B depict SDS-PAGE images showing the results of Nluc-HhC precursor protein purification. FIG. 10A depicts Nluc-HhC-His6 precursor protein purification. Key: (I) Nluc-HhC (44 kDa). FIG. 10B depicts Nluc-(D46H) HhC-SUMO-His6 precursor protein purification. Key: (II) Nluc-(D46H) HhC-SUMO-His6 (58 kDa), (III) (D46H) HhC-SUMO-His6 (39 kDa). The presence of (III) results from spontaneous self-cleavage (hydrolysis) of the autoprocessing domain, HhC (D46H)-SUMO, fragment from Nluc.

FIG. 11 depicts reversed phase HPLC chromatogram of the sterol modified oligonucleotide with 3′ quencher prepared by EDC-based sterylation. Sterol modified oligonucleotides (starred).

FIG. 12 depicts 1H NMR of the synthetic sterol amine (5).

FIG. 13 depicts 13C NMR of the synthetic sterol amine (5).

FIG. 14 depicts LC-MS analysis of the synthetic sterol amine (5).

FIG. 15 depicts reversed phase HPLC chromatogram of the sterol free (lower) and mono-sterylated oligonucleotide (upper) prepared by solid phase using Expedite 8909.

FIG. 16 depicts an image of the agarose gel for E-beacon purification. Nucleic acid moieties in the agarose gel were visualized by GelRed® nucleic acid gel stain giving bright bands.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

### DETAILED DESCRIPTION

In embodiments, the present disclosure relates to kits, compositions, or one or more methods of detecting an analyte-of-interest using one or more protein-oligonucleotide reporters of the present disclosure, such as an E-beacon. As used herein the term E-beacon, or Eb, refers to a protein-oligonucleotide reporters of the present disclosure. In embodiments, the one or more protein-oligonucleotide reporters of the present disclosure includes one or more conjugate reaction products such as a composition, including: a conjugate reaction product of: a signal emitting peptide or functional fragment thereof having a glycine amino acid residue; a linker characterized as a fused sterol or stanol ring system, having a nucleophilic group at a 3-position of an A-ring of a fused sterol or stanol ring system with beta or alpha stereochemistry; and an oligonucleotide having a 5′ end and a 3′ end, wherein the composition is characterized by one of the 3′ end or the 5′ end attached to the linker and one of the 3′ end or the 5′ end attached to a quencher, and wherein the linker is covalently linked to the predetermined electrophilic residue.

Advantages of the biosensors of the present disclosure such as one or more protein-oligonucleotide reporters or E-beacons include enhanced sensitivity, specificity and speed of isothermal amplification techniques such as NASBA and RT-LAMP. In embodiments, the biosensors, E-Beacons, or one or more protein-oligonucleotide reporters of the present disclosure mitigate obstacles by using an enzymatically generated turn-on light signal for an “all-in-one” homogenous and high-throughput testing platform.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

The term “about”, as used herein, refers to +/−20% of the stated value or a chemical or obvious equivalent thereof.

As used herein, the term “forming a mixture” refers to the process of bringing into contact at least two distinct species such that they mix together and interact. “Forming a reaction mixture” and “contacting” refer to the process of bringing into contact at least two distinct species such that they mix together and can react, either modifying one of the initial reactants or forming a third, distinct, species, a product. It should be appreciated, however, the resulting reaction product can be produced directly from a reaction between the added reagents or from an intermediate from one or more of the added reagents which can be produced in the reaction mixture. “Conversion” and “converting” refer to a process including one or more steps wherein a species is transformed into a distinct product.

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer including purine and pyrimidine bases or other natural, chemically or biochemically modified, non-natural, or derivatized nucleotide bases. The terms “polynucleotide” and “nucleic acid” should be understood to include, as applicable to the embodiments being described, single-stranded (such as sense or antisense) and double-stranded polynucleotides.

In embodiments, the term “oligonucleotide” refers to a polynucleotide of between 4 and 100 nucleotides of single- or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and can be isolated from genes, transcribed (in vitro and/or in vivo), or chemically synthesized.

The term “complementary” refers to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G), or in any other manner that allows for the formation of stable duplexes. Complementarity is typically measured with respect to a duplex region and thus excludes, for example, overhangs. A duplex region may include a region of complementarity between two strands or between two regions of a single strand, for example, a unimolecular siRNA. Typically, the region of complementarity results from Watson-Crick base pairing. In embodiments, perfect complementarity or 100% complementarity refers to the situation in which each nucleotide unit of one polynucleotide strand or region can hydrogen bond with each nucleotide unit of a second polynucleotide strand or region. Less than perfect complementarity refers to the situation in which one or more, but not all, nucleotide units of two strands or two regions can hydrogen bond with each other. For example, for two 20-mers, if only two base pairs on each strand can hydrogen bond with each other, the polynucleotide strands or regions exhibit 10% complementarity. In the same example, if 18 base pairs on each strand or each region can hydrogen bond with each other, the polynucleotide strands exhibit 90% complementarity. Substantial complementarity refers to polynucleotide strands or regions exhibiting 80% or greater complementarity.

By “hybridizable” or “complementary” or “substantially complementary” a nucleic acid (e.g. RNA, DNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA) base pairing with a guide RNA) is considered complementary to both a U and to C. For example, when a G/U base-pair can be made at a given nucleotide position of a protein-binding segment (e.g., dsRNA duplex) of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to one or more protein-oligonucleotide reporters binding to an analyte-of-interest such as a target nucleic acid, and the like) refers to a non-covalent interaction between macromolecules. While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶ M, less than 10⁻⁷ M, less than 10⁻⁸ M, less than 10⁻¹¹ M, less than 10⁻¹² M, or less than 10⁻¹⁵ M. “Affinity” refers to the strength of binding, increased binding affinity being correlated with a lower K_{d}.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains includes alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains includes lysine, arginine, and histidine; a group of amino acids having acidic side chains includes glutamate and aspartate; and a group of amino acids having sulfur containing side chains includes cysteine and methionine. Exemplary conservative amino acid substitution groups are: valine-to-leucine or isoleucine, phenylalanine-to-tyrosine, lysine-to-arginine, alanine-to-valine, and asparagine-to-glutamine.

As used herein the “degree of identity” refers to the relatedness between two amino acid sequences or between two nucleotide sequences and is described by the parameter “identity”. In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence. In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety.

In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm, and the sequence identity is calculated by identifying the number of exact matches identified by the program divided by the “alignment length”, where the alignment length is the length of the entire alignment including gaps and overhanging parts of the sequences.

The term “deoxynucleotide” refers to a nucleotide or polynucleotide lacking an OH group at the 2′ or 3′ position of a sugar moiety, and/or a 2′,3′ terminal dideoxy, but instead having a hydrogen at the 2′ and/or 3′ carbon.

The terms “deoxyribonucleotide” and “DNA” refer to a nucleotide or polynucleotide including at least one ribosyl moiety that has an H at the 2′ position of a ribosyl moiety. In embodiments, a deoxyribonucleotide is a nucleotide having an H at its 2′ position.

cDNA: The term “complementary deoxynucleotide” or “cDNA” means a DNA molecule that can be prepared by reverse transcription from a mature, spliced, mRNA molecule obtained from a eukaryotic or prokaryotic cell. cDNA lacks introns or intron sequences that may be present in corresponding genomic DNA. In embodiments, cDNA may refer to a nucleotide sequence that correspond to the nucleotide sequence of an mRNA from which it is derived. In embodiments, cDNA refers to a double-stranded DNA that is complementary to and derived from mRNA.

The term “isolated” means a substance in a form or environment that does not occur in nature. In embodiments, one or more protein-oligonucleotide reporters may be synthetically produced and are considered isolated for purposes of the present disclosure, as are native or one or more oligonucleotides of the present disclosure, which have been separated, fractionated, or partially or substantially purified by any suitable technique.

The term “nucleotide” refers to a ribonucleotide or a deoxyribonucleotide or modified form thereof, as well as an analog thereof. Nucleotides include species that include purines, e.g., adenine, hypoxanthine, guanine, and their derivatives and analogs, as well as pyrimidines, e.g., cytosine, uracil, thymine, and their derivatives and analogs. In embodiments, a “nucleotide” includes a cytosine, uracil, thymine, adenine, or guanine moiety. In embodiments, nucleotides, unless otherwise specified (such as, for example, when specifying a 2′ modification, 5′ modification, 3′ modification, nucleobase modification, or modified internucleotide linkage), include unmodified cytosine, uracil, thymine, adenine, and guanine. In embodiments, nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety as defined herein. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides. In embodiments, modified bases refer to nucleotide bases such as, for example, adenine, guanine, cytosine, thymine, and uracil, xanthine, inosine, and queuosine that have been modified by the replacement or addition of one or more atoms or groups. Some examples of types of modifications that can include nucleotides that are modified with respect to the base moieties, include but are not limited to, alkylated, halogenated, thiolated, aminated, amidated, or acetylated bases, in various combinations. More specific modified bases include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles. The term nucleotide is also meant to include what are known in the art as universal bases. By way of exampl′, universal bases include but are not limited to 3-nitropyrrole, 5-nitroindole, or nebularine. Further, the term nucleotide also includes those embodiments or species that have a detectable label, such as for example a radioactive or fluorescent moiety, or mass label attached to the nucleotide.

The phrase “nucleotide unit” refers to a single nucleotide residue and is includes a modified or unmodified nitrogenous base, a modified or unmodified sugar, and a modified or unmodified moiety that allows for linking of two nucleotides together or a conjugate that precludes further linkage.

As used herein, the terms “isolated nucleic acid fragment”, and “isolated nucleic acid molecule” are used interchangeably and are optionally single-stranded or double-stranded with synthetic, non-natural or modified nucleotide bases. This will indicate a single-stranded RNA or DNA polymer.

As used herein, the term “nucleic acid molecule” refers to any molecule containing multiple nucleotides (i.e., molecules comprising a sugar (e.g., ribose or deoxyribose) linked to a phosphate group and to an exchangeable organic base, which is either a substituted pyrimidine (e.g., cytosine, thymine (T) or uracil (U)) or a substituted purine (e.g., adenine (A) or guanine (G)). As described further below, bases include A, T, U, C, and G, as well as variants thereof. As used herein, the term refers to ribonucleotides (including oligoribonucleotides (ORN)) as well as deoxyribonucleotides (including oligodeoxynucleotides (ODN)). The term shall also include polynucleosides (i.e., a polynucleotide minus the phosphate) and any other organic base containing polymer. Nucleic acid molecules can be obtained from existing nucleic acid sources (e.g., genomic or cDNA), but include synthetic (e.g., produced by oligonucleotide synthesis). In embodiments, the terms “nucleic acid molecule” and “polynucleotide” may be used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

The term “substantially purified” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

As used herein, “fluorescent group” refers to a molecule that, when excited with light having a selected wavelength, emits light of a different wavelength. Fluorescent groups may also be referred to as “fluorophores”.

As used herein, “fluorescence-modifying group” refers to a molecule that can alter in any way the fluorescence emission from a fluorescent group. A fluorescence-modifying group generally accomplishes this through an energy transfer mechanism. Depending on the identity of the fluorescence-modifying group, the fluorescence emission can undergo a number of alterations, including, but not limited to, attenuation, complete quenching, enhancement, a shift in wavelength, a shift in polarity, a change in fluorescence lifetime. One example of a fluorescence-modifying group is a quenching group.

As used herein, “quenching group” refers to any fluorescence-modifying group that can attenuate at least partly the light emitted by a fluorescent group or fluorescent peptide. We refer herein to this attenuation as “quenching”. Hence, illumination of the fluorescent group or fluorescent peptide in the presence of the quenching group leads to an emission signal that is less intense than expected, or even completely absent. Quenching occurs through energy transfer between the fluorescent group or fluorescent peptide and the quenching group.

As used herein, “fluorescence resonance energy transfer” or “FRET” refers to an energy transfer phenomenon in which the light emitted by the excited fluorescent group is absorbed at least partially by a fluorescence-modifying group. If the fluorescence-modifying group is a quenching group, then that group can either radiate the absorbed light as light of a different wavelength, or it can dissipate it as heat. FRET depends on an overlap between the emission spectrum of the fluorescent group and the absorption spectrum of the quenching group. FRET also depends on the distance between the quenching group and the fluorescent group. Above a certain critical distance, the quenching group is unable to absorb the light emitted by the fluorescent group, or can do so only poorly.

As used herein, “3′ end” means at any location on the oligonucleotide from and including the 3′ terminus to the center of the oligonucleotide, usually at any location from and including the 3′ terminus to about 10 bp from the 3′ terminus, and more usually at any location from and including the 3′ terminus to about 5 bp from the 3′ terminus.

As used herein, “5′ end” means at any location on the oligonucleotide from and including the 5′ terminus to the center of the oligonucleotide, usually at any location from and including the 5′ terminus to about 10 bp from the 5′ terminus, and more usually at any location from and including the 5′ terminus to about 5 bp from the 5′ terminus.

As used herein the “signal emitting polypeptide” refers to an ATP-independent luciferase that utilizes a coelenterazine analog substrate (such as, furimazine) to produce high intensity, glow-type luminescence. The signal emitting polypeptides include, but are not limited to other luciferase enzymes that produce light, such as, for example, Firefly luciferase and Gaussia luciferase. These enzymes catalyze the oxidation of a specific substance or substrate, such as furimazine in the case of NanoLuc, which leads to the generation of photons. As used herein, “signal emitting polypeptide” and “light emitting polypeptide” are used interchangeably.

As used herein, with respect to the light emitting polypeptide “or function fragment thereof,” the functional fragment thereof is an engineered version of the ATP-independent luciferase (e.g., NanoLuc) in which the luciferase enzyme protein is split into two non-functional polypeptides. There can be a re-association of the two non-functional polypeptides of the luciferase to reconstitute the enzymatic activity leading to the generation of photons.

As used herein “a predetermined electrophilic residue” refers a specific glycine amino acid residue of the light emitting polypeptide, located at the final residue position of the light emitting polypeptide.

As used herein the “light emitting polypeptide” is inclusive of “light emitting peptide” located at the final residue position of the light emitting polypeptide. As used herein, a peptide is a short chain of amino acids that is typically from about 2 to about 50, linked by peptide bonds. A longer chain of linked amino acids, such as about 51 or more is a polypeptide.

As used herein the term a “protein-nucleic acid fusion molecule” refers to a biomolecular chimera, wherein a protein is connected covalently to a DNA or RNA molecule. The covalent connector used herein is a sterol molecule. These chimeras can be created in the lab using the hedgehog method, as detailed in U.S. Pat. No. 10,738,338 to Callahan et al. (herein entirely incorporated by reference). As disclosed herein, the chimera is comprised of ATP-independent luciferase linked to a sterol molecule, which is linked to a hairpin structured DNA oligonucleotide.

As used herein the term a “protein-oligonucleotide reporters” are oligonucleotides referred to as “detector” strands since they are preselected to bind to nucleic acid of a specific sequence of interest via hybridization to form an oligonucleotide reporters complex, or duplex.

As used herein the term “amplicon” refers to a piece of single stranded or double stranded DNA or RNA or DNA/RNA duplex that is the source and/or product of amplification or replication events. It can be formed artificially, using various methods including polymerase chain reactions (PCR) or ligase chain reactions (LCR), or naturally through gene duplication.

Any given component, or combination of components can be unlabeled, or can be detectably labeled with a label moiety. In some cases, when two or more components are labeled, they can be labeled with label moieties that are distinguishable from one another.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### Detailed Description of Certain Embodiments

In embodiments, the present disclosure relates to a composition, including: a peptide reporter molecule, and a hairpin-forming mono-sterylated oligonucleotide including a quencher. In embodiments, the peptide reporter molecule is a light emitting polypeptide or light emitting enzyme such as the Nluc enzyme or a functional fragment thereof. In embodiments, Nluc enzyme, luciferase, or functional fragments thereof generate a signal such as light when contacted under suitable conditions with an indicator or substrate such as furimazine or coelenterazine. However, light generated by the peptide reporter molecule may be quenched when in close proximity to a fluorescence-modifying group, or quencher group. Non-limiting examples of a quencher group includes dabcyl, lowa Black, or combinations thereof. In embodiments, a peptide reporter molecule of the present disclosure is characterized as isolated, substantially purified, or both.

In embodiments, the present disclosure relates to binding one or more protein-oligonucleotide reporters to a predetermined nucleic acid to change the shape of the one or more protein-oligonucleotide reporters from a first conformation to a second conformation, wherein the second conformation generates or bolsters a signal such as a light or luminescent signal. In embodiments, the present disclosure provides radiation emitting compositions suitable for generating a signal such as light useful or indicative of the predetermined nucleic acid such as an analyte-of-interest. In embodiments, the signal amplifies or bolsters another signal.

Non-limiting examples of analytes-of-interest include a predetermined nucleic acid complementary or substantially complementary to a segment of the compositions of the present disclosure such as the hairpin-forming mono-sterylated oligonucleotide. In embodiments, the hairpin-forming mono-sterylated oligonucleotide is preselected to be complimentary to an analyte-of-interest. Non-limiting examples of analytes-of-interest include pathogenic nucleic acids, nucleic acids from one or more aberrant cells, one or more predetermined nucleic acids, one or more viral RNA's, or one or more viral DNA's.

In embodiments, viral nucleic acid-of-interest includes a single or partially double stranded viral DNA, or viral RNA. In some embodiments, viral nucleic acid-of-interest includes viral ribonucleic acid-of-interest and/or viral deoxyribonucleic acid-of-interest to be detected and/or identified in accordance with the methods of the present disclosure. Non-limiting example of ribonucleic acid-of-interest include RNA from one or more riboviruses, or RNA from one or more RNA viruses. Non-limiting examples of RNA viruses include virus that causes the common cold, influenza virus, SARS virus, SARS-CoV-2, Dengue Virus, hepatitis C virus, hepatitis E virus, West Nile fever virus, Ebola virus, rabies virus, polio virus and measles virus. In some embodiments, RNA viruses include those in which The International Committee on Taxonomy of Viruses (ICTV) classifies as RNA viruses such as those that belong to Group III, Group IV or Group V of the Baltimore classification system of classifying viruses and does not consider viruses with DNA intermediates in their life cycle as RNA viruses. In embodiments, viruses with RNA as their genetic material which also include DNA intermediates in their replication cycle, retroviruses, and include Group VI of the Baltimore classification such as HIV-1 and HIV-2 may also be identified in accordance with the methods of the present disclosure. In some embodiments, the ribonucleic acid of interest may include ribonucleic acid from double stranded RNA viruses, single stranded plus sense RNA viruses or single stranded negative sense RNA viruses.

Non-limiting examples of deoxyribonucleic acid-of-interest include DNA from one or more DNA viruses. Non-limiting DNA viruses may include poxviruses such as avipox virus, myxoma virus or vaccinia virus; herpes simplex virus (HSV), cytomegalovirus (CMV), Epstein-Barr virus (EBV), hepadna virus (e.g. hepatitis B) Viruses), herpes viruses such as polyoma virus, papilloma virus, adenovirus and adeno-associated virus; or single-stranded DNA viruses such as parvovirus.

In embodiments, the compositions and methods of the present disclosure are suitable for detecting the presence of viral nucleic acid such as viral DNA or viral RNA based on using one or more protein-oligonucleotide reporters such as E-beacons designed to undergo a conformational change (from looped to linear) upon binding a target viral nucleic acid or binding to an analyte-of-interest. In embodiments, the presence of the viral nucleic acid, such as viral RNA would be indicated by increased luminescence formed as the linear conformation separates a quencher from a light emitting polypeptide or functional fragment thereof. In embodiments, the hairpin-forming mono-sterylated oligonucleotide segment of the peptide-oligonucleotide reporter is preselected to be complementary to the analyte-of-interest such that the hairpin segment will bind to the analyte-of-interest, when present.

In some embodiments, the present disclosure relates to a method of determining a presence of an analyte-of-interest, including: contacting a composition including a light emitting polypeptide or functional fragment thereof, a linker characterized as a fused sterol or stanol ring system, and an oligonucleotide having a 5′ end attached to the linker and a 3′ end attached to a quencher with an analyte of interest under conditions sufficient to separate the light emitting polypeptide or functional fragment thereof from the quencher to generate a signal. For example, one or more methods of detecting an analyte-of-interest such as an RNA virus includes reconfiguring one or more protein-oligonucleotide reporters by contacting the one or more protein-oligonucleotide reporters having a first conformation characterized as “off”′ with a biological specimen to form a mixture, wherein when the mixture includes an analyte-of-interest, the first conformation changes to a second conformation characterized as “on”; and reacting the second conformation, when present, with an indicator such as furimazine under conditions sufficient to form a signal.

In embodiments, one or more protein-oligonucleotide reporters are preformed or preselected to include a hairpin forming segment that will combine with a preselected analyte-of-interest such as e.g., a preselected nucleic acid, a preselected ribonucleotide-of-interest, a preselected RNA, or preselected RNA oligonucleotide. In embodiments, the preselected nucleic acid is single stranded.

In embodiments, one or more protein-oligonucleotide reporters include an enzyme or peptide portion and an oligonucleotide portion, wherein the oligonucleotide portion has a preselected segment suitable for hybridizing with a preselect analyte-of-interest. In embodiments, analytes-of-interest, or targets are detected based on their interactions with the one or more protein-oligonucleotide reporters and the conformational changes that are induced in the one or more protein-oligonucleotide reporters as result of such interactions. In embodiments, the one or more protein-oligonucleotide reporters are designed so that in the absence of the target they typically maintain a looped or hairpin (or off) conformation and assume a linear (or on) conformation in the presence of an analyte-of-interest such as a target. These conformations are detected as the peptide portion and oligonucleotide portion including a quencher physically separate or form a linear conformation. Where a quencher is positioned at a sufficient distance from the peptide, a signal will be generated. For example, a light signal will be formed when the light emitting polypeptide such as Nluc enzyme or luciferase separates from a quencher under suitable conditions such as in the presence of a substrate like furimazine, coelenterazine or luciferin. In embodiments, signal such as light informs that the analyte-of-interest or target has altered the conformation of the one or more protein-oligonucleotide reporters.

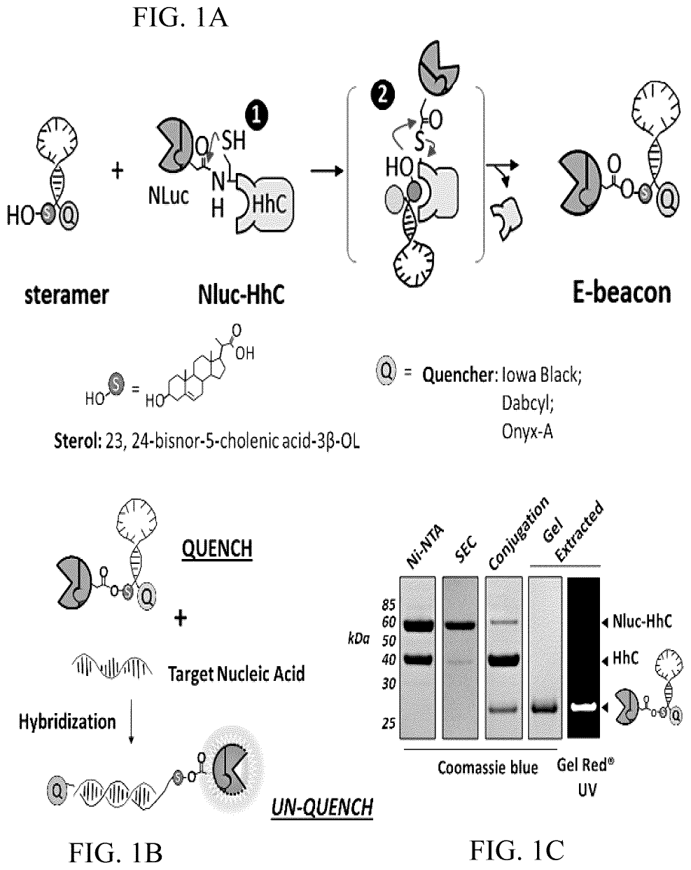

Embodiments of the present disclosure are shown in the figures including FIGS. 1A-1C. As noted above, FIGS. 1A-1C depict a method of the present disclosure and biocatalytic preparation of a composition of the present disclosure. FIG. 1A depicts a scheme showing HhC catalyzed bioconjugation of Nluc (grey) to a hairpin-forming mono-sterylated oligonucleotide (steramer) modified with a 3′ dark quencher (Q). In embodiments, conjugation involves formation of an internal thioester (step 1), followed by steramer binding and acyl transfer to the sterol hydroxyl group (step 2). Referring now to FIG. 1B, a general mechanism is shown for turn-on analyte-of-interest, or nucleic acid detection using a composition of the present disclosure, where hybridization of the Nluc conjugated hairpin oligonucleotide with complementary nucleic acid displaces the quencher (Q) from Nluc, enhancing bioluminescence signal. FIG. 1C depicts a preparation of a composition of the present disclosure monitored by SDS-PAGE. A fusion of Nluc with HhC is purified first by Ni-NTA chromatography, followed by size exclusion chromatography (SEC), then reacted with steramer (conjugation).

Composition of the present disclosure such as E-beacon is isolated by agarose gel extraction. Nucleic acid was visualized by UV with GelRed stain.

In embodiments, one or more protein-oligonucleotide reporters of the present disclosure are designed to detect and/or identify viral RNA targets. For example, in embodiments, one or more protein-oligonucleotide reporters are configured to bind to a binding partner to alter the shape of the one or more protein-oligonucleotide reporters, e.g., viral RNA. In embodiments, the binding partner may be one or more preselected ribonucleic acids, e.g. viral RNA, or fragments thereof that bind(s) to the one or more protein-oligonucleotide reporters and open the one or more protein-oligonucleotide reporters into a linear conformation. In embodiments, the preselected nucleic acid is RNA or DNA based on sequence complementarity. In embodiments, and as shown in FIG. 1B, one or more protein-oligonucleotide reporters of the present disclosure bind to a pre-selected target nucleic acid and change the conformation of the one or more protein-oligonucleotide reporters to a linear, unquenched, or “on” conformation. The linear conformation presence is indicated by increased fluorescence.

In embodiments, detection of one or more analytes-of-interest such as viral RNA analytes-of-interest is important for a variety of applications including for example in the fields of medicine and forensics. In some embodiments, the present disclosure provides a programmable peptide-oligonucleotide reporter that undergoes a pre-defined conformational change upon contact with an analyte-of-interest such as a target ribonucleic acid such as viral RNA, converting a protein-oligonucleotide reporters from a looped or “off” state to a linear “on” state (or conformation) within a peptide-oligonucleotide reporter-nucleic acid complex.

In embodiments, the linear “on” state relates to a nucleic acid complex or conformation that includes a combination of the peptide-oligonucleotide reporter and a preselected nucleic acid or target nucleic acid combined to form a peptide-oligonucleotide reporter-nucleic acid complex having a second conformation, wherein the second conformation is characterized as linear, unquenched and/or “on”. See FIG. 1B. In embodiments, a peptide-oligonucleotide reporter and/or peptide-oligonucleotide reporter nucleic acid complex can be detected using visual inspection or use of a luminometer.

In embodiments, the one or more protein-oligonucleotide reporters of the present disclosure are stable in complex fluids such as but not limited to serum-containing samples. In some embodiments, the protein-oligonucleotide reporters for use herein are configured to convert from unbound to bound forms in the presence of complex fluids. Moreover, the protein-oligonucleotide reporters are also stable for an extended period of time. Once synthesized, the protein-oligonucleotide reporters may be frozen and stored for days, weeks or months. Similarly, protein-oligonucleotide reporters complexes of the present disclosure formed when the protein-oligonucleotide reporters bind with the analyte-of-interest may be stable for an extended period of time. Once synthesized, the protein-oligonucleotide reporters-complexes may be frozen and stored for days, weeks or months.

In some embodiments, the protein-oligonucleotide reporters of the present disclosure can be made using chemical synthetic routes suitable for binding or fusing nucleic acids to peptides or proteins such as but not limited to the methodology set forth in U.S. Pat. No. 10,738,338 to Callahan et al. (herein entirely incorporated by reference). The chemistry described therein is suitable for forming a conjugate reaction product of: a signal emitting peptide or functional fragment thereof having a glycine amino acid residue located at the final residue position of the light emitting polypeptide; a linker characterized as a fused sterol or stanol ring system, having a nucleophilic group at a 3-position of an A-ring of a fused sterol or stanol ring system with beta or alpha stereochemistry; and an oligonucleotide having a 5′ end and a 3′ end, wherein the composition is characterized by one of the 3′ end or the 5′ end attached to the linker and one of the 3′ end or the 5′ end attached to a quencher, and wherein the linker is covalently linked to the glycine amino acid residue. Moreover, the examples below describe, among other things, how to make the conjugate reaction products and reporters of the present disclosure.

In embodiments, the conjugation product includes an oligonucleotide having a 5′ end and a 3′ end as set forth above. In embodiments, oligonucleotide having a 5′ end and a 3′ end suitable for use herein may be of any length sufficient to allow association (i.e., binding) and dissociation (i.e., unbinding) of binding partners to occur and to be distinguished from other association and/or dissociation events using signal generated in accordance with the present disclosure. In embodiments, the oligonucleotide having a 5′ end and a 3′ end is at least 10 nucleotides in length, and it may be as long as 1,000 nucleotides in length (or it may be longer). The oligonucleotide having a 5′ end and a 3′ end may therefore be 10-500 nucleotides in length, 10-100 nucleotides in length, 10-200 in length, or any range therebetween. In some embodiments, the oligonucleotide having a 5′ end and a 3′ end ranges in length from about 20-35 nucleotides and may be a predetermined or preselected length to facilitate binding with a preselected or predetermined analyte-of-interest. In embodiments, the length of oligonucleotides will depend in part on the application, the length of the analyte-of-interest, and the length of the oligonucleotides themselves. In embodiments, the oligonucleotides are designed to be of approximately equal length. In some embodiments, the oligonucleotides may be about 20-100 nucleotides in length. The oligonucleotides may be, without limitation, about 20, about 30, about 40, about 50, about 60, about 70, about 80, about 90 or about 100 nucleotides in length. In some embodiments, the oligonucleotides may be about 40-80 nucleotides in length. In some embodiments, the oligonucleotides may be about 60 nucleotides in length.

In some embodiments, the oligonucleotide having a 5′ end and a 3′ end may be a naturally occurring nucleic acid. In some embodiments, the oligonucleotide having a 5′ end and a 3′ end may also be non-naturally amplified nucleic acids such as polymerase chain reaction (PCR)-generated nucleic acids, rolling circle amplification (RCA)-generated nucleic acids, etc. In some embodiments, the oligonucleotide having a 5′ end and a 3′ end is rendered single-stranded either during or post synthesis. Methods for generating a single-stranded oligonucleotide having a 5′ end and a 3′ end include asymmetric PCR. Alternatively, double-stranded nucleic acids may be subjected to strand separation techniques in order to obtain the single-stranded scaffold nucleic acids. In embodiments, the oligonucleotide having a 5′ end and a 3′ end may include DNA, RNA, DNA analogs, RNA analogs, or a combination thereof, provided it is able to hybridize in a sequence-specific and non-overlapping manner to the analyte-of-interest.

In embodiments, the oligonucleotide having a 5′ end and a 3′ end may be synthetic, and/or include a hairpin forming segment that is complimentary to the analyte-of-interest. In embodiments, the oligonucleotides may be characterized as modified or unmodified or variable oligonucleotides. An example is a variable oligonucleotide including a phosphate at their 5′ end (referred to herein as a 5′ phosphate). Oligonucleotides having this latter modification are used herein in the detection of target nucleic acids, and in this context such oligonucleotides are referred to as “detector” strands since they are preselected to bind to nucleic acids of the interest via=having a linear conformation, wherein the linear conformation is characterized as unquenched or “on”.

In embodiments, and as described above, one or more protein-oligonucleotide reporters may be pre-selected to hybridize to the one or more analytes-of-interest and form a peptide-oligonucleotide reporter complex of the present disclosure. In the embodiments, the one or more analytes-of-interest include a ribonucleic acids-of-interest such as an RNA, viral RNA, or a combination thereof. In embodiments, the ribonucleic acid-of-interest may be a naturally occurring ribonucleic acid, or one or more fragments thereof. In embodiments, the ribonucleic acid-of-interest is a positive-sense, single-stranded RNA from SARS-COV-2.

In embodiments, the analyte-of-interest such as a ribonucleic acid-of-interest, refers to the nucleic acid that is hybridized to the peptide-oligonucleotide reporter. It is to be understood that the analyte-of-interest may derive from and thus be a fragment of a much larger nucleic acid such as for example viral RNA. Thus, a binding portion of the analyte-of-interest (i.e., the ribonucleic acid bound to the protein-oligonucleotide reporters) may range from about 7-50 nucleotides, or e.g., 10 to 35 nucleotides, or 5 to 30 nucleotides in some instances, while its parent ribonucleic acid may be much longer (for example on the order to kbs or more).

In some embodiments, the conditions that allow an analyte-of-interest such as a ribonucleic acid-of-interest such as viral RNA to hybridize to the oligonucleotide segment of the peptide-oligonucleotide reporter may be standard hybridization conditions as known in the art. Such conditions may include a suitable concentration of salt(s) and a buffer. The condition may also include EDTA in order to preserve the protein-oligonucleotide reporters complex.

In some embodiments, the hybridization may be accomplished using a constant annealing temperature. Such constant temperature may range from about 4° C. to 55° C., 15° C. to 30° C., or 20° C. to 30° C., or may be about 25° C. The temperature may be regarded as room temperature (RT). The hybridization may be carried out over a period of hours such as 1, 2, 3, 4, 5 hours or more depending on the application.

In some embodiments, the hybridization may be accomplished by decreasing the temperature from a temperature at which the analyte-of-interest such as a ribonucleic acid-of-interest and the oligonucleotide of the peptide-oligonucleotide reporter are not hybridized to each other to a temperature at which they are hybridized to each other. This is referred to herein as a temperature ramp or a decreasing annealing temperature. The starting temperature may be about 40-60° C. without limitation. The ending temperature may be about 4-25° C. without limitation. Thus, the temperature ramp may be from about 50° C. to about 4° C. or about 40° C. to about 4° C. In embodiments, a temperature ramp from about 46° C. to about 4° C. The change in temperature is typically carried out over 1-12 hours. Thus, the change in temperature may decrease by about 0.1-1° C. per minute.

Regardless of whether a constant or decreasing annealing temperature is used, the hybridization may also be carried out for much shorter periods of time, for example on the order of 10−30 minutes, provided readout can be achieved. Thus, in some instances, if the method determines if the nucleic acid-of-interest is present and has formed a peptide-oligonucleotide reporter-ribonucleic acid complex of the present disclosure, then the hybridization period can be short, particularly if the ribonucleic acid-of-interest such as viral RNA is present in abundance. In some embodiments, longer hybridization times may be required. Similarly, if the analyte-of-interest such as a ribonucleic acid-of-interest is present in low abundance, longer hybridization times may be required, particularly if an amplifying latch mechanism is used. In some embodiments, only a portion or preselected portion of the ribonucleic acid-of-interest hybridizes to the reporter molecule of the present disclosure.

In embodiments, the present disclosure includes a composition, including: a conjugate reaction product of: a signal emitting peptide or functional fragment thereof having a glycine amino acid residue located at the final residue position of the light emitting polypeptide; a linker characterized as a fused sterol or stanol ring system, having a nucleophilic group at a 3-position of an A-ring of a fused sterol or stanol ring system with beta or alpha stereochemistry; and an oligonucleotide having a 5′ end and a 3′ end, wherein the composition is characterized by one of the 3′ end or the 5′ end attached to the linker and one of the 3′ end or the 5′ end attached to a quencher, and wherein the linker is covalently linked to the glycine amino acid residue. In embodiments, the oligonucleotide is characterized as a single-stranded nucleic acid. In embodiments, the oligonucleotide is DNA or RNA. In embodiments, the oligonucleotide comprises a hairpin loop. In embodiments, the reaction product has a first conformation in an absence of a predetermined target strand and a second conformation in a presence of a predetermined target strand, if any. In embodiments, the reaction product further includes a predetermined target strand hybridized to the oligonucleotide. In embodiments, the signal emitting peptide is a luciferase enzyme. In embodiments, the signal emitting peptide is an enzyme or functional fragment thereof. In embodiments, the quencher is a dark fluorescence quencher. In embodiments, the oligonucleotide has a first segment and a second segment and a third segment, wherein the first and second segment are complementary and form a self-annealed stem structure and the third segment forms a loop that is preselected to hybridize to a preselected analyte of interest. In embodiments, the oligonucleotide is preselected to hybridize to a preselected analyte of interest. In embodiments, the oligonucleotide is preselected to hybridize to viral DNA or viral RNA. In embodiments, the oligonucleotide has a first confirmation characterized as closed and, when contacted with a preselected nucleic acid, forms a second conformation characterized as signal emitting.

In embodiments, the present disclosure includes a composition, including:

a peptide reporter molecule, and a hairpin-forming mono-sterylated oligonucleotide comprising a quencher. In embodiments, the peptide reporter molecule is a light emitting polypeptide or light emitting enzyme.

In embodiments, the present disclosure includes a method of making a composition, including: contacting a light emitting polypeptide or functional fragment thereof having an a glycine amino acid residue located at the final residue position of the light emitting polypeptide with a linker characterized as a fused sterol or stanol ring system having a nucleophilic group at a 3-position of an A-ring of a fused sterol or stanol ring system with beta or alpha stereochemistry, and an oligonucleotide having a 5′ end and a 3′ end or the 5′ end or the 3′ end attached to the quencher. In embodiments, the light emitting polypeptide or functional fragment thereof is an enzyme.

In embodiments, the present disclosure includes a method of determining a presence of an analyte of interest, including: contacting a composition comprising a light emitting polypeptide or functional fragment thereof, a linker characterized as a fused sterol or stanol ring system, and an oligonucleotide having a 5′ end attached to the linker and a 3′ end attached to a quencher with an analyte of interest under conditions sufficient to separate the light emitting polypeptide or functional fragment thereof from the quencher to generate a signal. In embodiments, the oligonucleotide is characterized as a single-stranded nucleic acid. In embodiments, the oligonucleotide is DNA or RNA. In embodiments, the oligonucleotide comprises a hair-pin loop. In embodiments, the oligonucleotide has a first segment, a second segment, and a third segment, wherein the first segment and second segment form a self-annealed stem structure and the third segment forms a loop that is preselected to hybridize to a preselected analyte of interest.

In embodiments, the present disclosure includes a method of reconfiguring a protein-nucleic acid fusion molecule, including: contacting a protein-nucleic acid fusion molecule, having a first conformation, with an oligonucleotide of interest to form a protein-nucleic acid fusion molecule complex having a second conformation, wherein the second conformation is characterized as on; and contacting the second conformation with an indicator under conditions sufficient to form a signal. In embodiments, the signal is predetermined to show a presence or absence of oligonucleotide of interest.

In embodiments, the present disclosure includes a composition, including: an enzyme-nucleic acid molecule including one or more nucleic acid binding sites, wherein the enzyme-nucleic acid molecule has a first conformation characterized as off, and a second conformation characterized as on when in a presence of an analyte-of-interest.

### Examples

Materials: All chemicals were obtained from commercial suppliers and used directly unless otherwise mentioned: Fos-Choline-12 (Anatrace); Imidazole (Acros Organics); Triethylammonium acetate (Calbiochem); Dimethyl sulfoxide (EMD Millipore Corp.); Acetonitrile, glycerol tris(2-carboxyethyl) phosphine, Tris Base (Fisher Scientific Inc.); MeOH (Macron Fine Chemicals); Ampicillin, Isopropyl β-d-1-thiogalactopyranoside (MP Biomedicals); Nano-Glo® Luciferase Assay System (Promega); N-(3-Dimethylaminopropyl)-N′-ethylcarbodiimide hydrochloride, n-butanol, LB agar (miller), Luria Bertani Broth (Sigma); 23, 24-BISNOR-5-CHOLENIC ACID-3β-OL (Steraloids); KCl, MgCl₂ (VWR). The CPG solid supports (3′-dabcyl-CPG or dT-CPG, 1.0 μM), DNA phosphoramidites (dT-CE, dABZ-CE, dCAc-CE and dGdmf-CE), 5′-carboxy modifier-C10 amidite, 0.25 M tetrazole in acetonitrile, capping and oxidation solutions (Glen Research).

Plates: Corning® 96 Well Black Polystyrene Microplate (#3650; dialysis chambers: EMD Millipore Corp. D-Tube™ Dialyzer Maxi, MWCO 12-14 kDa (#71510); and concentrators: Corning® Spin-X® UF 6 mL Centrifugal Concentrator, 5,000 MWCO Membrane. (#431482).

Buffers: Bacterial Cell Lysis buffer: 0.5% Triton X-100, 0.05 M K₂HPO₄, 0.4 M NaCl, 0.1 M KCl, 10% glycerol, 0.01 M imidazole, pH=7.3; Ni-NTA Bind buffer: 1 M NaCl, 0.04 M Na₂HPO₄, 0.06 M imidazole, 20% glycerol, pH=7.5; Ni-NTA Elution buffer: 0.02 M Na₂HPO₄, 0.5 M NaCl, 0.5 M imidazole, 10% glycerol, pH=7.3; agarose gel extraction buffer: 20 mM Tris HCl, pH=7.4; and Luciferase Assay DNA hybridization buffer: 100 mM KCl, 1 mM MgCl₂, 10 mM Tris HCl, pH=8.0.

Methods-protein expression/purification: Two C-terminal His-tagged Nluc-HhC precursor constructs were used. The first construct Nluc-HhC has been described previously (See e.g., Zhang, X.; Xu, Z.; Moumin, D. S.; Ciulla, D. A.; Owen, T. S.; Mancusi, R. A.; Giner, J. L.; Wang, C.; Callahan, B. P., Protein-Nucleic Acid Conjugation with Sterol Linkers Using Hedgehog Autoprocessing. Bioconjug Chem 2019, 30 (11), 2799-2804). The second construct, Nluc-HhC (D46H)-SUMO, differs in using a gain-of-function HhC mutant along with a SUMO tag for enhanced expression and solubility (See e.g., Zhao, J.; Ciulla, D. A.; Xie, J.; Wagner, A. G.; Castillo, D. A.; Zwarycz, A. S.; Lin, Z.; Beadle, S.; Giner, J. L.; Li, Z.; Li, H.; Banavali, N.; Callahan, B. P.; Wang, C., General Base Swap Preserves Activity and Expands Substrate Tolerance in Hedgehog Autoprocessing. J Am Chem Soc 2019, 141 (46), 18380-18384). E. coli BL21 (DE3) containing expression plasmid for each His-tagged Nluc-HhC precursor was grown at 37° C. in 50 ml of LB broth with carbenicillin (100 μg/ml) and 250 RPM shaking. Once OD600 reached 0.6-0.8, IPTG was added to the culture (0.5 mM, final) to induce expression. After 18-20 hours at 16° C., bacterial cells were harvested by centrifugation at 10,000 RPM for 10 mins, spent media was discarded and the pellet was resuspended in bacterial lysis buffer (3 ml). After 3 freeze (−80° C.)/thaw cycles and sonication, insoluble material was removed by centrifugation at 10,000 RPM for 60 min. To the clarified lysate, an equal volume of ice chilled 2× Ni-NTA bind buffer was added. The solution was applied to Ni-NTA spin columns (Cytiva). His-tagged precursor was purified according to the manufacture's protocol. Purified Nluc-HhC or Nluc-HhC (D46H)-SUMO precursor protein was stored at −80° C. in Ni-NTA elution buffer with added TCEP (5× 10-3 μM).

See for example, FIGS. 10A and 10B depicting SDS-PAGE images and the results of Nluc-HhC precursor protein purification. FIG. 10A depicts Nluc-HhC-His6 precursor protein purification. Key: (I) Nluc-HhC (44 kDa). FIG. 10B depicts Nluc-(D46H) HhC-SUMO-His6 precursor protein purification. Key: (II) Nluc-(D46H) HhC-SUMO-His6 (58 kDa), (III) (D46H) HhC-SUMO-His6 (39 kDa). The presence of (III) results from spontaneous self-cleavage (hydrolysis) of the autoprocessing domain, HhC (D46H)-SUMO, fragment from Nluc.

Solution-based EDC oligonucleotide sterylation (used for EB.1-3):

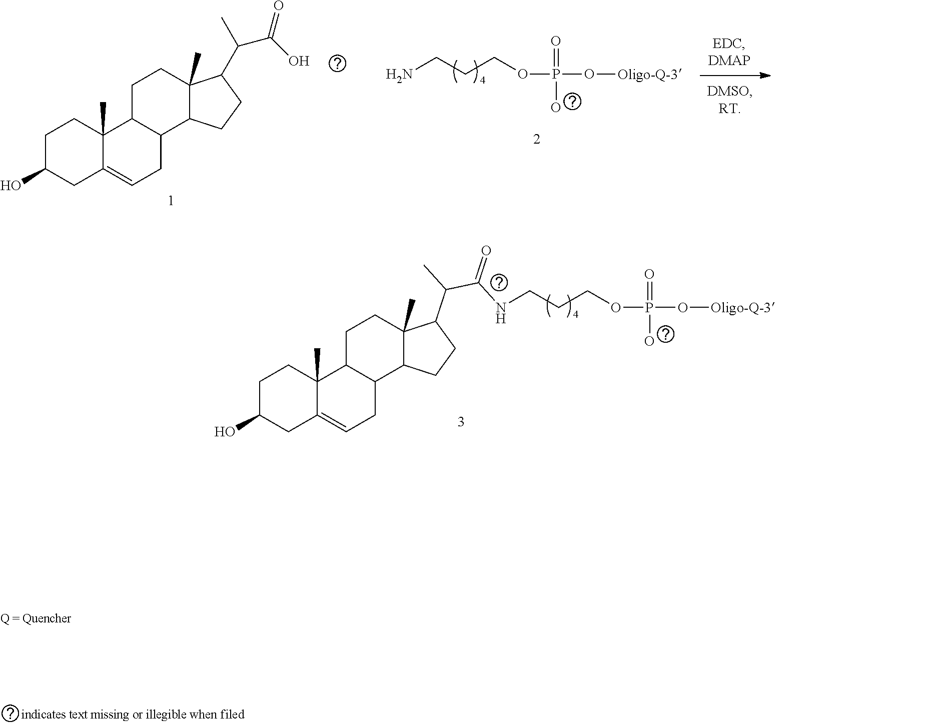

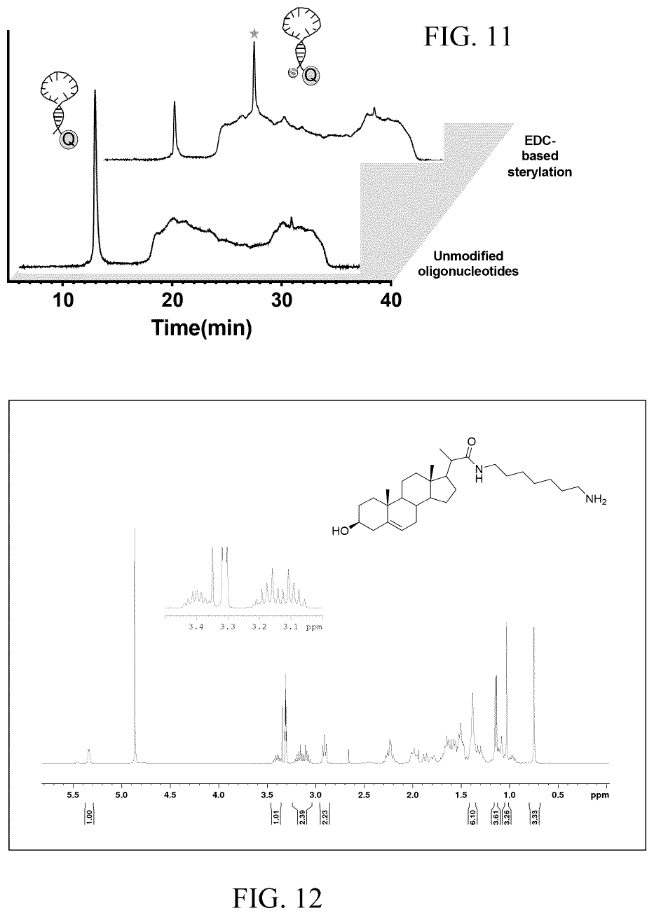

4.33 mg (12.5 μmol) of 23, 24-bisnor-5-cholenic acid-3-ol (1) and 23.95 mg (125 μmol) of N-(3-Dimethylaminopropyl)-N′-ethylcarbodiimide hydrochloride (EDC) was added to 200 μl of DMSO. After incubation at room temperature for 30 min, 1.5 mg (12.5 μmol) of 4-Dimethylaminopyridine (DMAP) was added and incubated at room temperature for another 5 min. Last, 50 μl of 100 UM 5′-amino modified oligo (2) (in H₂O) was added and the solution was vortexed gently at room temperature overnight. The coupling reaction was extracted by adding 20 μl of 3 M sodium acetate, pH 5.2, and 1 ml of n-butanol. After a brief vortex and incubation at −80° C. for 1 hour, oligonucleotide was collected as a precipitate by centrifugation at 14,000 RPM for 20 min. The pellet was resuspended in 50 μl of water. Sterylated oligonucleotide (steramer) (3) was separated from sterol-free oligonucleotide by RP-HPLC, see e.g., FIG. 11 depicting reversed phase HPLC chromatogram of the sterol modified oligonucleotide with 3′ quencher prepared by EDC-based sterylation. Sterol modified oligonucleotides (starred).

### Synthesis and purification of sterol amine (5) for solid phase oligo coupling (for EB.19s)

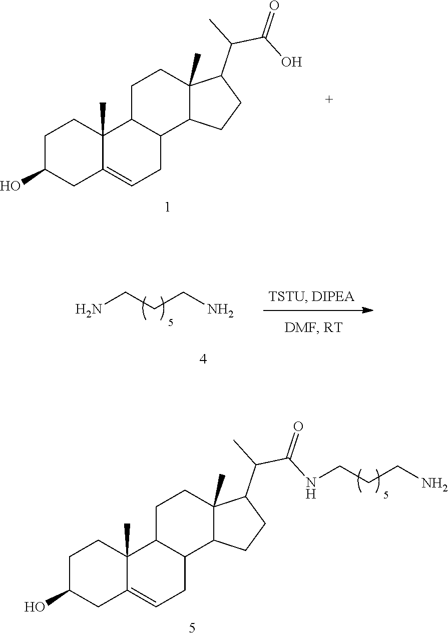

173.5 mg (0.5 mmol) of 23, 24-bisnor-5-cholenic acid-3B-ol (1), 435 μl (2.5 mmol) of N,N-Diisopropylethylamine (DIPEA), and 165.6 mg (0.55 mmol) of N,N,N′, N′-Tetramethyl-O-(N-succinimidyl) uronium tetrafluoroborate (TSTU) was added to 6 ml of anhydrous DMF. The mixture was stirred at room temperature for 30 minutes. After that the mixture was added to 2 ml of 0.75 M 1,7-Diaminoheptane (4) in anhydrous DMF dropwise. Then the final reaction mixture was stirred at room temperature for 20 hours. After that the reaction mixture was washed with dichloromethane (10 ml) and saturated NaHCO₃ solution (10 ml) for three times, dried over Na₂SO₄, and evaporated under N₂ flow to afford 167.7 mg crude sterol amine (5) as a white to yellow solid.

The crude sterol amine (5) was dissolved in 1 ml of methanol and purified over a Restek viva C18 5 μm HPLC column (250×4.6 mm) using a gradient elution from 0% acetonitrile to 100% acetonitrile over 25 minutes. The flow rate was 1 mL/min and eluate was monitored at 210 nm. Sample injection volume was 100 μl. After HPLC purification, the solvent was evaporated to afford sterol amine (5) (144.6 mg, 63%) as a white solid.

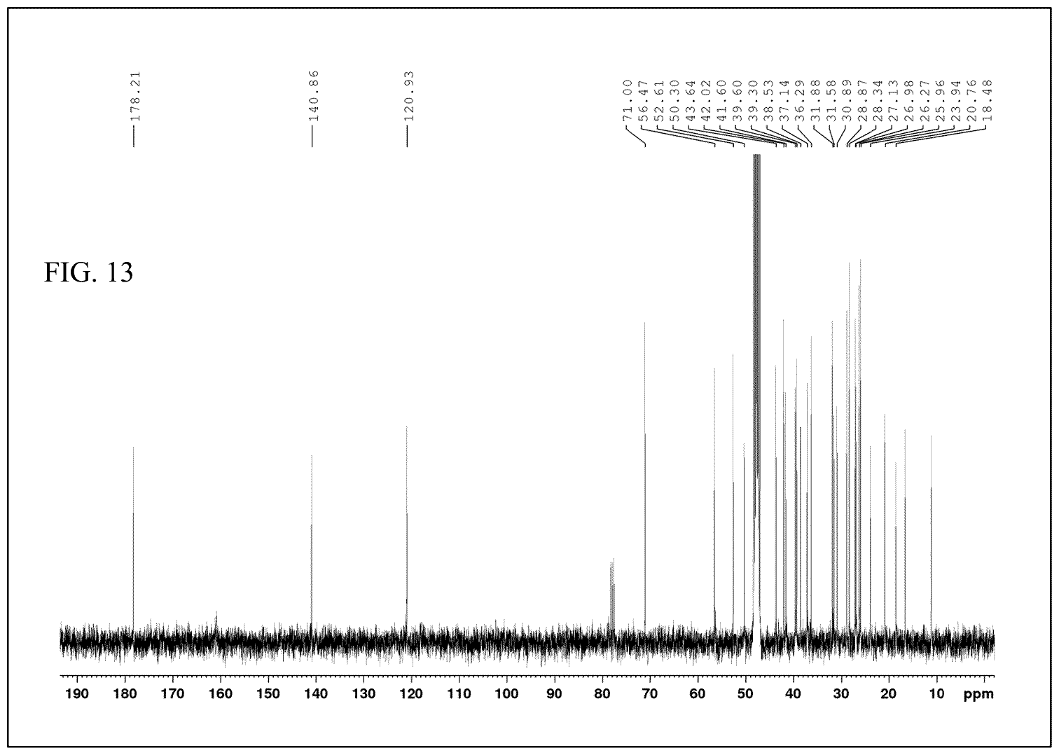

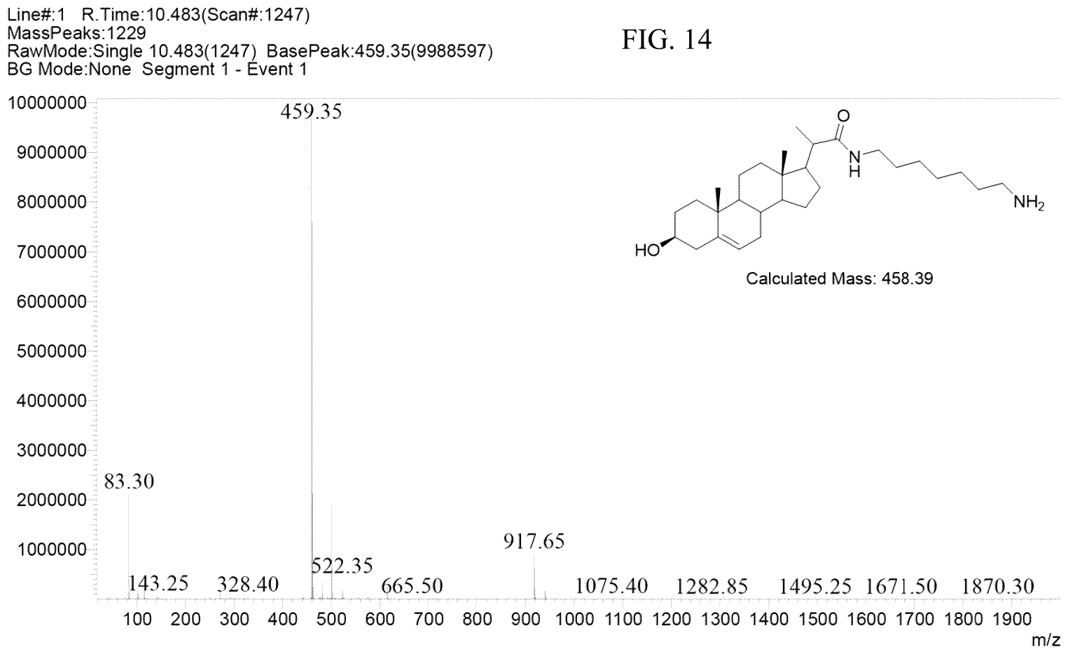

NMR Characterization of (5): 1H NMR spectra were acquired with Bruker Avance III HD 400 (400 MHZ) spectrometer at 25° C. CD₃OD (Sigma) was used as NMR solvent. 1H chemical shifts are reported as δ in units of parts per million (ppm) relative to methanol-d (3.31, s) 1H NMR (400 MHZ, CD₃OD) δ 5.34 (dd, 1H), 3.40 (m, 1H), 3.16 (m, 1H), 3.11 (m, 1H), 2.91 (t, 2H), 2.17 (dd, 1H), 1.38 (s, 6H), 1.14 (d, 2H), 1.03 (s, 3H), 0.75 (s, 3H). 13C NMR (100 MHz, CD₃OD) δ 178.21, 140.86, 120.93, 71.00, 56.47, 52.61, 50.30, 43.64, 42.02, 41.60, 39.60, 39.30, 38.53, 37.14, 36.29, 31.88, 31.58, 30.89, 28.87, 28.34, 27.13, 26.98, 26.27, 25.96, 23.94, 20.76, 18.48, 16.56, 11.09. See e.g., FIG. 12 depicting 1H NMR of the synthetic sterol amine (5); FIG. 13 depicting 13C NMR of the synthetic sterol amine (5); and FIG. 14 depicting LC-MS analysis of the synthetic sterol amine (5).

Solid Phase Steramer Synthesis: The sterylated-DNA oligomers (steramers, Table 1) were synthesized on a 1.0 μM scale (3′-dabcyl-CPG or dT-CPG) using standard DNA synthesis protocols on an automated Expedite 8909 DNA/RNA synthesizer. After synthesizing the desired or preselected DNA sequence, the 5′-end was coupled with 5′-carboxy modifier-C10 carrying a reactive NHS ester. The final detritylation step was eliminated after coupling with the 5′-carboxy modifier-C10. Prior to the next coupling with sterol amine, the solid support was washed with acetonitrile followed by drying with nitrogen purging for 5 minutes.

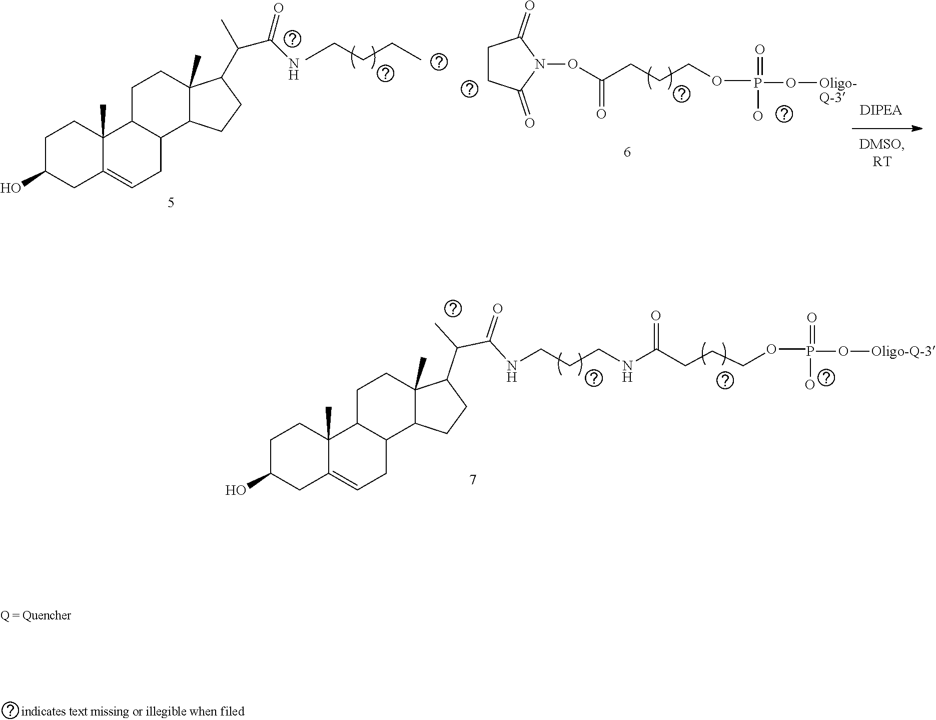

The manual coupling of sterol amine (5) with the oligo NHS ester (6) on the CPG solid support was performed in anhydrous DMSO (0.05 M in 5) containing 10% DIPEA at room temperature for 2 hours using the push-pull syringe method. Sterol amine 5 (21 mg, 45 mmole) was dissolved in 810 mL of dry DMSO followed by the addition of 90 mL of dry DIPEA to make 900 mL of coupling cocktail. 200 ml of the above coupling cocktail was used for each 1 mmole synthesis. After the coupling, the column was thoroughly washed with 5×1 mL of acetonitrile and dried.

Steramers (7) were cleaved from the solid support using 40% methyl amine in water (2×0.8 mL) at room temperature for 2 hours. Lyophilization of the cleavage solution gave the crude steramers. Exposure to light was avoided while handling the 3′-dabcyl labeled steramers. The identity of all the synthesized steramers was confirmed by the MALDI-TOF mass analysis (Table 1). Crude samples of all the steramers showed ˜85-90% conversion by RP-HPLC (see Figure S12).

Supporting Table 1: List of steramers prepared by solid phase synthesis

| | | Mass |
|---|---|---|
| E-beacons | *Sequence (5′-3′) | calcd./found |
| Eb.2′ | sterol-CGCTC CCAAAAAAAAAAACC | 8780/8783 |
| | GAGCG(SEQ ID NO: 1)-dabcyl | |
| Eb.19 (E484) | sterol-CGCTC TGGTGTTGAAGGTTT | 8909/8912 |
| | GAGCG(SEQ ID NO: 2)-dabcyl | |
| Eb.19 (E484K) | sterol-CGCTC TGGTGTTAAAGGTTT | 8893/8898 |
| | GAGCG(SEQ ID NO: 3)-dabcyl | |

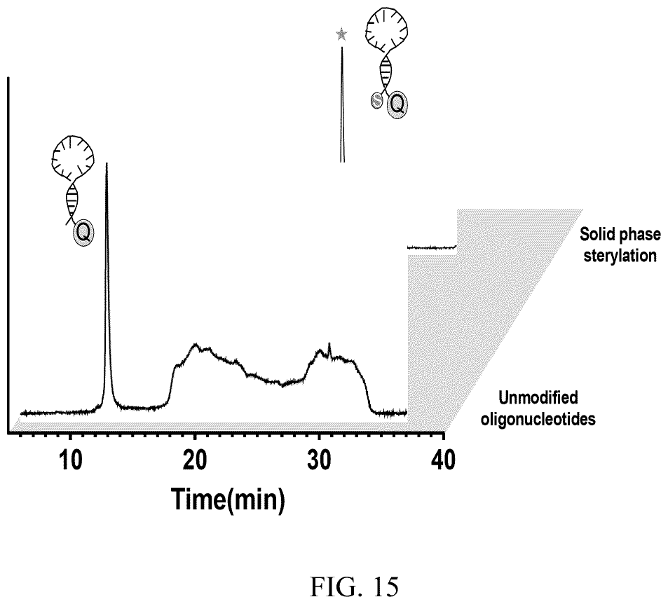

* The underlined regions self-anneal to form the hairpin stem. The nucleotides in black bold font were for the target RNA/DNA detection, e.g., one or more preselected RNA or DNA targets. Sterol moiety at the 5′-end was introduced for the bioconjugation with nanoluciferase and the dabcyl unit at 3′-end was as a dark fluorescence quencher. See also, FIG. 15 depicting reversed phase HPLC chromatogram of the sterol free (lower) and mono-sterylated oligonucleotide (upper) prepared by solid phase using Expedite 8909.

General Conditions for E-beacon bioconjugation by HhC: To prepare Nluc-hairpin nucleic acid conjugates, Nluc-HhC precursor protein (2×10⁻⁶ M, final), Fos-choline 12 (1.5×10⁻³ M, final), Tris(2-carboxyethyl) phosphine hydrochloride (TCEP, 5×10⁻³ M, final), Bis-Tris buffer (0.05 M, final, pH 7.1), ethylenediaminetetraacetic acid (EDTA, 5×10⁻⁴ M, final), NaCl (0.1 M, final) were mixed. To that solution, Steramer (1×10⁻⁴ M, final) was added and the reaction was incubated at 16° C. overnight.

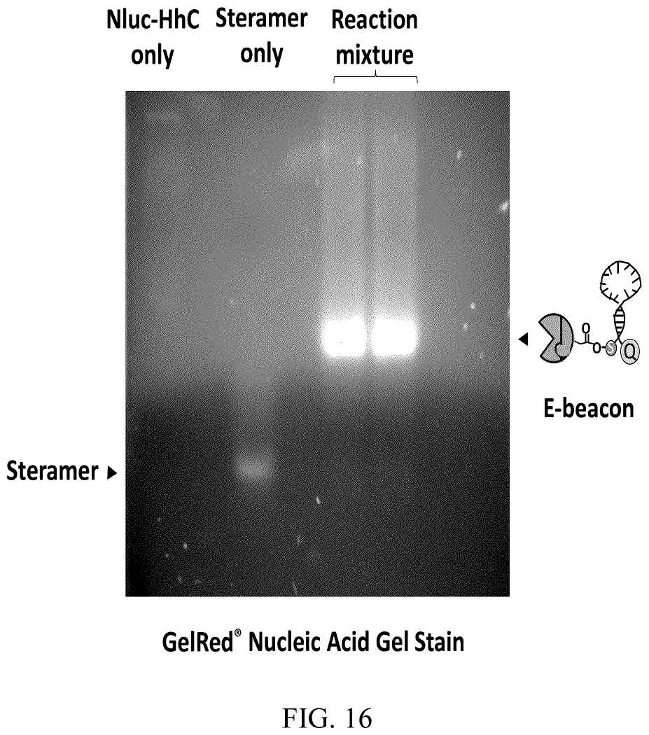

E-beacon isolation by agarose gel extraction: Agarose gel extraction was used as a rapid means of isolating E-beacon. E-beacon conjugation reaction (100 μl) was combined with 6X gel loading buffer (20 μl) and separated on 2% agarose gel containing GelRed® Nucleic Acid Gel Stain (followed the manufacturer's “precast protocol”). The gel was run at 90 V in 1× TAE buffer until the sample loading dye front was approximately 95% toward the end of the gel. E-beacon conjugate was visualized with BioRad Gel Doc® (UV tray), then excised, diced, transferred to an Eppendorf tube and soaked in Tris buffer (3 ml, 20 mM pH7.4) at 4° C. overnight. After centrifugation to gel fragment, the supernatant was concentrated by a Spin-X® UF Concentrator (Corning), 5 kDa MWCO. See e.g., FIG. 16 depicting an image of the agarose gel for E-beacon purification. Nucleic acid moieties in the agarose gel were visualized by GelRed® nucleic acid gel stain giving bright bands.

E-beacon Bioluminescence Measurements: To perform luminescence reading, 25 μl of E-Beacon solution was combined with 25 μl of sample nucleic acid in the DNA hybridization buffer a Corning® 96 Well Black Polystyrene Microplate. After incubation at 25° C. for the selected interval, we added 25 μl of Nano-Glo® Luciferase Assay System substrate (Promega). After another 5 min, bioluminescence was measured using a Synergy H1 Hybrid Multi-Mode Microplate Reader (BioTek).

DICUSSION: Reagents for the specific detection of dilute nucleic acid are fundamental to molecular and cellular genetics and various clinical diagnostics, including tests for viral pathogens like SARS-COV-2. Molecular beacons, the fluorogenic hairpin-forming oligonucleotide-based sensors, have provided a standard detection tool. Modified with fluorophore and quencher at opposite ends, the oligonucleotide fluorescence is suppressed in the hairpin, or off state. Molecular beacons switch on by hybridizing to complementary nucleic acid, which separates the fluorophore and quencher, increasing radiative emission. Improvements to molecular beacon technology have been driven mainly through chemical synthesis with the introduction of more efficient quenchers and new fluorophore/quencher pairs.

Embodiments of the present disclosure include an alternative detection strategy where biocatalysis is employed in the preparation of the sensor and for the sensor output. In enzymatic beacons or E-beacons the fluorophore of a molecular beacon is replaced by the compact, ATP-independent bioluminescent enzyme, nanoluciferase (Nluc). This substitution provides an internal, amplifiable light source. Nluc with the engineered substrate furimazine produces light that is sufficiently bright for measurement using a portable luminometer. With detection signal enzymatically amplified, we also expected that nucleic acid detection assays would consume less reagent while improving sensitivity relative to synthetic molecular beacons.

To connect Nluc to a hairpin-forming oligonucleotide as a potential E-beacon, the protein-nucleic acid bioconjugation activity found in hedgehog precursor proteins (FIG. 1A) was applied. Hedgehog precursor proteins harbor a promiscuous C-terminal autoprocessing domain, HhC, that catalyzes two linked activities: self-cleavage from an adjacent N-terminal protein, and site-specific sterol ligation to that departing N-terminal protein. These reactions occur without cofactor or NTP requirements. Drosophila melanogaster HhC has broad substrate tolerance, maintaining robust bioconjugation activity toward heterologous N-terminal substrate proteins and catalyzing ligation with sterols of varying structure and chemical appendages, including oligonucleotides. For E-beacons, the promiscuous HhC was fused to the C-terminus of the Nluc enzyme (See methods above).

To serve as an HhC substrate for ligation to Nluc, the hairpin oligonucleotide component required mono-sterylation. The initial E-beacon preparations used a SSDNA oligo with the sequence: (sterol)-5′-CGCTCCCAAAAAAAAAAACCGAGCG (SEQ ID NO:1)-(3′-IBQ). The underlined regions self-anneal to form the hairpin stem; the italicized 15 nucleotides represent the probe or loop region for target nucleic acid hybridization. The same sequence was used by Kramer, Tyagi et al. for their early biophysical studies on molecular beacons. The lowa Black (IBQ) dark quencher was initially used at the (3′) to suppress Nluc bioluminescence at the (5′) in the hairpin (off) state (See FIG. B). Mono-sterylation (5′) was achieved by coupling a (5′) alkyl amine modifier of the oligo to the carboxyl group of 23, 24-bisnor 5-cholenic acid-3B-ol (See e.g., FIG. 1A, and methods above).

E-beacon, Eb.1, was prepared by combining the Nluc-HhC precursor protein with mono-sterylated hairpin-forming oligodeoxynucleotide in vitro. [15] His-tagged Nluc-HhC precursor was expressed in E. coli and purified in soluble form by Ni-NTA resin and size exclusion chromatography (FIG. 1C, lanes 1-2). Site-specific bioconjugation of Nluc to the sterylated oligo was carried out as described by Zhang et al. In a typical reaction, Nluc-HhC precursor was incubated at 2 μM (final) with 25-50-fold excess of mono-sterylated oligo in HhC buffer, overnight on the benchtop, then assessed reaction progress by SDS-PAGE (FIG. 1C, see conjugation). Agarose gel extraction provided a convenient means of purifying the conjugate, Eb.1 (FIG. 1C, last two lanes).

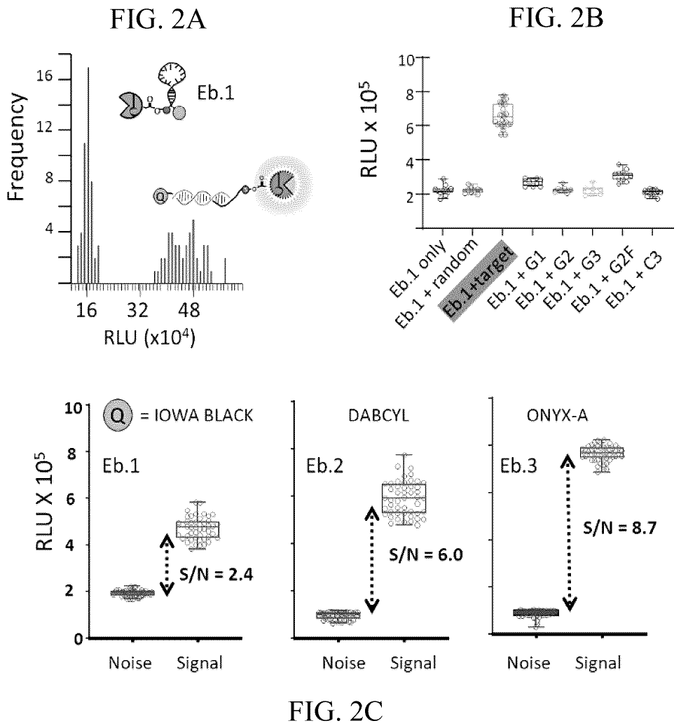

Nucleic acid sensing with Eb.1 was observed. As an initial test, bioluminescence readings from samples of Eb.1 with added oligonucleotide complementary to the hairpin probe region (signal; GGTTTTTTTTTTTGG (SEQ ID NO: 4)) were collected and compared with bioluminescence readings from Eb. 1 mixed with non-complementary oligonucleotide (noise; CTGGTCTTCGGGCTA (SEQ ID NO: 5)). In these experiments, Eb.1 was present at 2×10⁻⁹ M and oligonucleotide was added to 25×10⁻⁹ M. Samples were incubated in hybridization buffer (KCl 100 mM, MgCl₂ 1 mM, in 10 mM Tris buffer, pH 8) at 25° C. for 30 min followed by addition of the Nluc substrate, furimazine, according to the manufacturer's instructions (Promega). Representative data from a 96-well experiment, with 48 signal and 48 noise samples, are summarized with the histogram in FIG. 2A. The signal/noise was 2.5-3-fold, comparing the ratio of Eb.1 samples with complementary oligo to Eb.1 samples with random oligo. The increased Eb.1 bioluminescence accords with the general unquenching mechanism: target/probe hybridization displaces the 3′-quencher from the light source, here Nluc, permitting brighter signal.

Next, the specificity of Eb. 1 as a nucleic acid reporter was more stringently evaluated by measuring bioluminescence output when combined with test oligos carrying only one to three mismatches with the hairpin probe. Oligo sequences are listed in Table 1. As above, each oligo was added to 25×10⁻⁹ M in hybridization buffer containing Eb.1 at 2×10⁻⁹ M. These experiments were carried out “blinded”: one lab member distributed complementary and mismatched oligonucleotides into sample wells of a 96-well plate in a semi-random pattern; another lab member without knowledge of the plate layout added Eb.1, recorded bioluminescence measurements and carried out the data analysis. The results of a representative E-beacon specificity test are summarized in FIG. 2B. The largest enhancement in Eb.1 bioluminescence is apparent in samples mixed with perfectly complementary target (signal/noise, 3-fold). No increase in bioluminescence was apparent in wells containing oligos with central mismatches of two and three base pairs (G2 and G3). The oligo with one mismatch (G1) in the center produced 10% unquenching of the E-beacon. Unquenching of 15-20% of Eb.1 was observed when mixed with oligo G2F, carrying (T-to-G) mismatches with the probe region at the 3′ and the 5′ flanks. Nonetheless, samples of Eb. 1 with G1 and G2F, compared with samples of Eb.1 plus the fully complementary oligo, were still easily distinguished. The fidelity of Eb.1 observed here suggested that this prototype's specificity, like a synthetic molecular beacon, was sufficiently strong for detecting subtle sequence variations.

Table 1. Sequences of hairpin oligodeoxynucleotides for E-beacons and ssDNA oligonucleotides used for evaluating E-beacon function. Except where indicated otherwise, sequences are shows from 5′ to 3′. Red=mismatch; Bold=codon 484 of SARS-COV-2 spike protein; [S] sterol; [Q] quencher

| Name | Sequence |
|---|---|
| Eb. 1-3 | [S]-CGCTC CCAAAAAAAAAAACC GAGCG(SEQ ID NO: 1)-[Q] |
| EB.1 Target: | GGTTTTTTTTTTTGG(SEQ ID NO:4) |
| Random: | CTGGTCTTCGGGCTA(SEQ ID NO:5) |
| G1: | GGTTTTGTTTTTTGG(SEQ ID NO:6) |
| G2: | GGTTTTGGTTTTTGG(SEQ ID NO:7) |
| G3: | GGTTTTGGGTTTTGG(SEQ ID NO:8) |
| G2F: | GGGTTTTTTTTTGGG(SEQ ID NO:9) |
| C3: | GGTTTTCCCTTTTGG(SEQ ID NO:10) |
| Eb. 19(WT) | 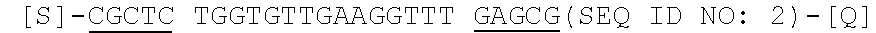 |
| E484 Target | (3') ACCACAACTTOCAAA(SEQ ID NO:11) (5') |
| Eb.19(E484K) | 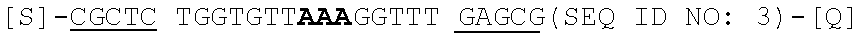 |
| E484K Target | (3') ACCACAATTTOCAAA(SEQ ID NO: 12) (5') |
| E484Q Target | (3') ACCACAAGTTCCAAA(SEQ ID NO: 13 (5') |

While encouraged by these results, the Eb.1 signal/noise of ˜3 and the assay Z′ factor of 0.46 and viewed as narrow and in need of improvement. The Z′ factor indicates assay robustness by the degree of separation between the values of positive (signal) and negative (noise) samples; the ideal Z′ factor is 1. A Z′ factor of 0.5 suggests that the assay reliability is borderline. This analysis led us to consider other quencher groups for the E-beacon. The peak bioluminescence from the Nluc/furimazine reaction occurs at 460 nm. The maximum absorbance of the lowa Black quencher is 531 nm. Better overlap was sought between the Nluc bioluminescence and the absorbance spectrum of the quencher. As alternative dark quenchers, we evaluated dabcyl and ONYX-A (Sigma). The dabcyl max absorbance is 453 nm and the ONYX-A absorbance peak is 515 nm. For meaningful comparison with Eb. 1, we prepared new E-beacons from mono-sterylated hairpin oligos carrying the same sequence as Eb.1 and we applied the same biocatalytic approach for site-specific attachment to the C-terminus of Nluc. The dabcyl containing E-beacon, Eb.2, and the ONYX-A E-beacon, Eb.3, were likewise isolated by agarose gel extraction.

Changing the E-beacon quencher improved signal/noise and assay quality, in the order ONYX-A>dabcyl>IBQ. FIG. 2C compares Eb.1 Eb.2 and Eb.3 readings with E-beacons at 2×10⁻⁹ M and complementary or random oligo at 25×10⁻⁹ M. Eb.3 with the ONYX-A quencher showed the highest single/noise of 8.7-fold and Z′ of 0.79. With the dabcyl quencher in Eb.2, the signal/noise was 6, better than IBQ, however the scatter in readings dropped the Z′ to 0.47. Because the hairpin sequences in EB.1-. 3 were the same, the changes in signal/noise are difficult to explain in terms of intra-oligo interactions, solution properties of the E-beacons, or Nluc activity; they seem more likely the result of specific photochemical characteristics (e.g. quenching efficiency). Structural information on SIGMA's ONYX quencher which might have helped interpret these results is not yet available.

Lastly and with a view toward application, E-beacons were made for SARS-COV-2 detection. Reliable assays for rapid identification of patients infected with SARS-COV-2, particularly those shedding viable virus, remain crucial to pandemic management. In considering an E-beacon configuration to facilitate scale-up for high-volume testing, the dabcyl was used rather than the ONYX-A or lowa Black quenchers. Dabcyl modified oligos are widely available and, unlike ONYX-A, dabcyl phosphoramidites can be purchased for solid phase oligo synthesis. With dabcyl, an oligonucleotide sterylation protocol was improved from microscale EDC-based solution coupling to a more robust solid phase coupling method with a DNA synthesizer. This approach routinely provided >90% yield for oligonucleotides carrying 5′ sterol and 3′ dabcyl modifications (See methods above). Because of the high yield and on-column washing steps, sterylated oligos prepared this way did not require additional HPLC purification prior to bioconjugation of Nluc by HhC. As an additional benefit, it was found that E-beacon incorporating solid-phase synthesized oligos proved superior over our initial dabcyl-quenched E-beacon, Eb.2, as measured by S/N and by Z′ factor. Compared to Eb.2, newly prepared dabcyl-quenched E-beacons showed an average S/N of 8-9, compared with 6 for Eb.2, and the assay results were more reliable, with a Z′ factor of 0.8 compared with 0.47 for Eb.2.

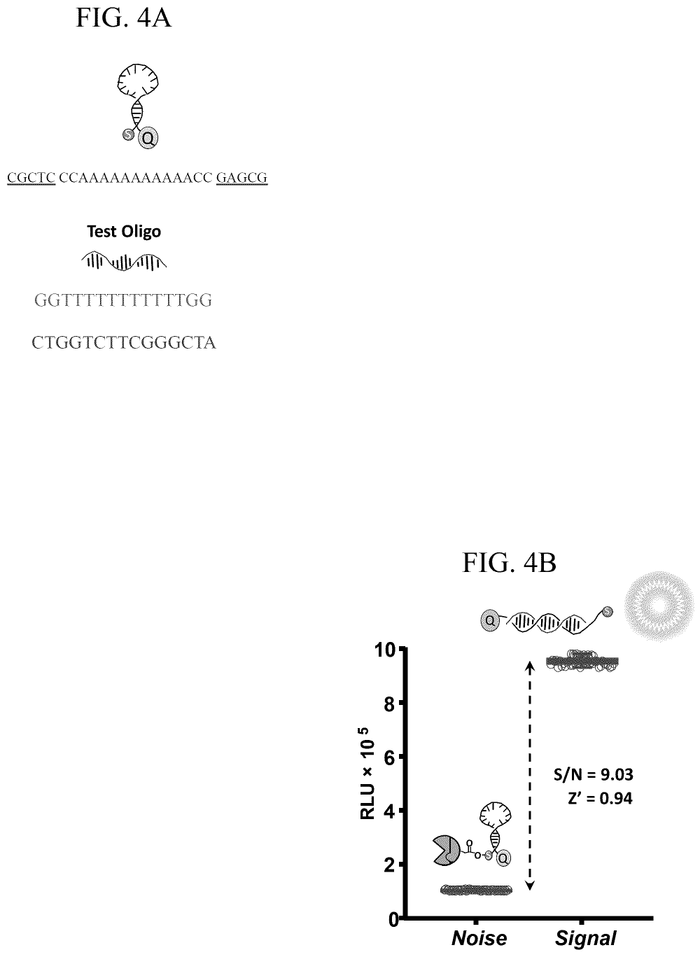

Referring now to FIGS. 4A and 4Bm the Spike coding region of SARS-CoV-2 was selected as target for the E-beacons of the present disclosure. The glutamate 484 codon of Spike was focused on as changes at this codon can help distinguish wild-type SARS-COV-2 from emerging variants. The E-beacon intended for wild-type, Eb. 19 (WT), was prepared by HhC catalyzed ligation of Nluc to the sterylated dabcyl-modified hairpin oligo: (sterol)-5′-CGCTCTGGTGTTGAAGGTTTGAGCG (SEQ ID NO: 2)-3′-(dabcyl). A second E-beacon was prepared for detecting the E484K mutation, an “escape mutation” which is present in certain a along with β, γ, I, η and μ variants of SARS-COV-2. This Eb. 19 (E484K) incorporated the oligo: (sterol)-5′-CGCTCTGGTGTTAAAGGTTTGAGCG (SEQ ID NO:3)-3′-(dabcyl). The bold text represents the spike 484 codon.

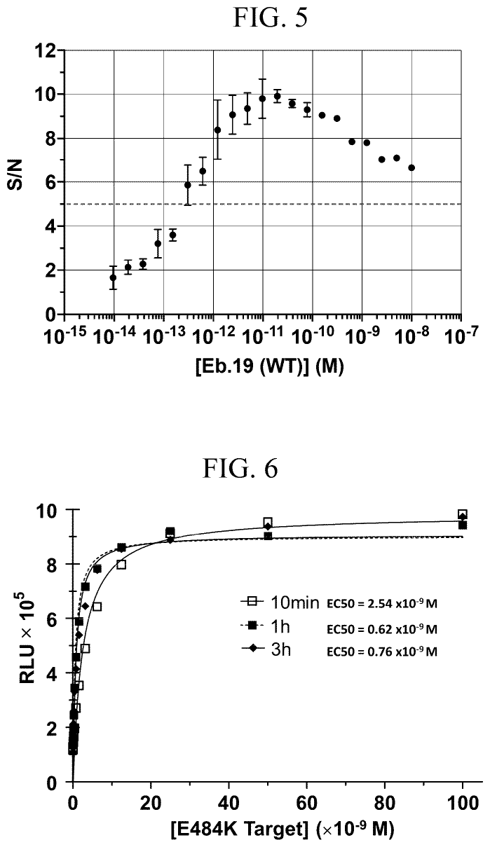

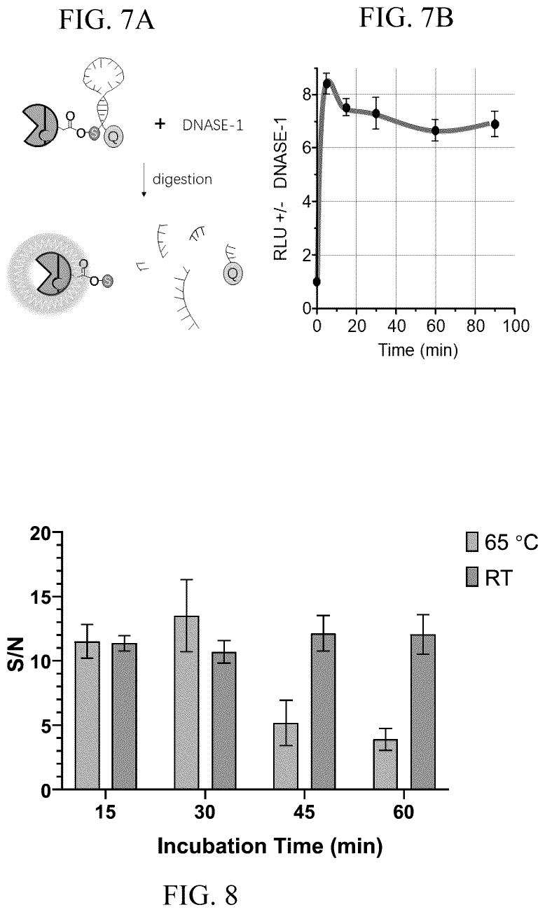

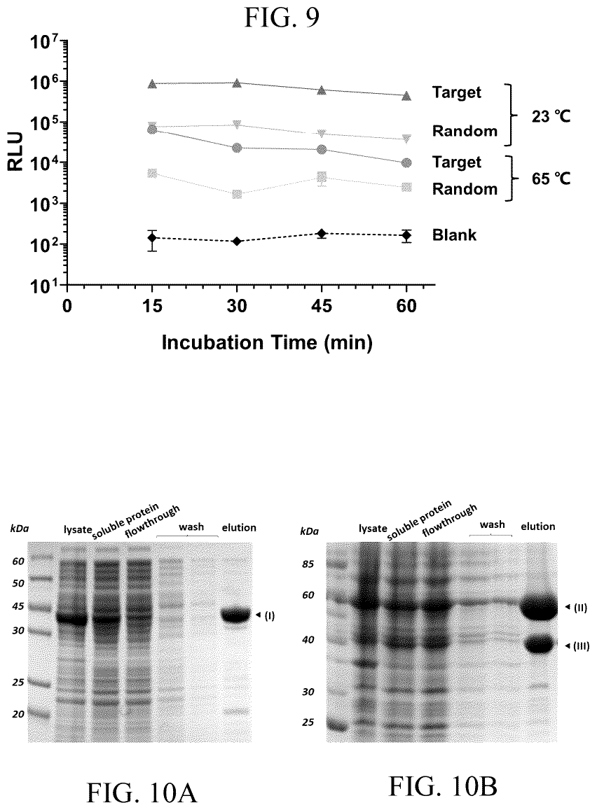

The potential assay conditions for the SARS-COV-2 E-beacons was explored in two stages. First, it was sought to determine the minimum working concentration of E-beacon. In these experiments, the concentration of target oligonucleotide, which is a DNA analog of the virus RNA, was held constant and in excess at 1×10⁻⁷ M, while the E-beacon was titrated from 1×10⁻⁸ M to 1×10⁻¹⁴ M. Bioluminescence readings following addition of substrate furimazine were recorded as before. A positive result was defined arbitrarily as having a signal/noise of >5 fold. With the S/N threshold at 5, the lowest possible operating concentration of Eb. 19 (WT) was 3×10⁻¹³ M (See e.g., FIG. 5). Measurements at this low concentration of E-beacon required the luminometer gain setting at the maximum level.

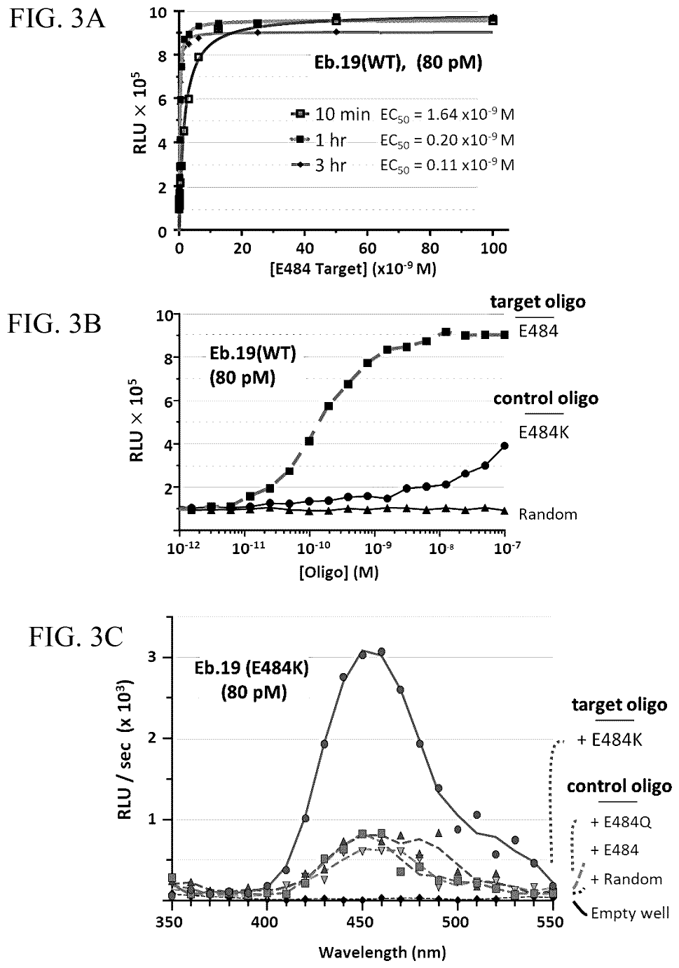

In stage two, the E-beacon concentration was held constant and the target oligos were titrated to find the detection threshold. 80×10⁻¹² M for was selected for the E-beacon. Although higher than the minimum Eb. 19 (WT) concentration defined above, 80 pM was selected to avoid pitfalls associated with assaying biomolecules in the extremely dilute regime, such as slow association kinetics and idiosyncratic adsorption effects, i.e., “death by dilution”. The assay parameter sought here was the EC₅₀ or the concentration of target oligo that could produce 50% un-quenching of Eb.19 (WT). The effect of E-beacon/target oligo incubation time on sensor output was explored. In FIG. 3A, bioluminescence readings of samples with Eb. 19 (WT) were shown plotted as a function of increasing target oligo concentration after 10 min, 1 hr and 3 hr incubations. Hyperbolic binding isotherms were fit to the experimental data to derive EC₅₀ values. For Eb. 19 (WT) the EC₅₀ was 1.64×10⁻⁹ M for the 10 min incubation; with the extended 1 hr and 3 hr incubations, the Eb. 19 (WT) appeared ˜10-fold more sensitive (FIG. 3A, inset). Similar behavior was observed with Eb.19 (E484K) (FIG. 6). The improved sensitivity (lower EC₅₀) with a 1-3 hr incubation of Eb19 (WT) and target oligo indicates that slow onset of E-beacon/target hybridization is a factor to consider with very dilute nucleic acid.

To assess specificity, bioluminescent signal from samples of Eb. 19 (WT) mixed with target oligo was compared with signal from samples of Eb. 19 (WT) mixed with the random oligo and the SARS-COV-2 E484K variant oligo (single base-pair mismatch, see Table 1). As with Eb.1, the random oligo did not unquench Eb. 19 (WT) over the concentration range tested (10⁻¹² to 10⁻⁷ M) (FIG. 3B, lowest trace). Unquenching of Eb. 19 (WT) did become apparent in samples containing the E484K oligo, although this “noise” required 1000-fold higher concentration of the E484K oligo over the target oligo (FIG. 3B, compare squares to circles). In FIG. 3C, selectivity data for Eb.19 (E484K) is presented in the form of comparative bioluminescence spectra. Here Eb.19 (E484K) was mixed with either E484K target oligo (FIG. 3C, top trace), with random oligonucleotide, E484 (WT) or an oligo corresponding to E484Q of the SARS-CoV-2 Kappa variant. Although some unquenching is apparent with the single base variants, signal of Eb.19 (E484K) was greatest with the fully complementary target, E484K (6-8 fold). Together, the results in FIGS. 3A-C are promising and show that with 80 pM E-beacon, detection is sensitive to 1 nM target nucleic acid and base pair specific.

In summary, the preparation and in vitro characterization of E-beacons, enzymatic bioluminescent counterparts to synthetic fluorogenic molecular beacons has been shown. In embodiments, E-beacons are provided by site-specific C-terminal coupling of nanoluciferase using HhC as the bioconjugation catalyst. This approach yields 1:1 enzyme: oligonucleotide stoichiometry. Protein-nucleic acid bioconjugation with HhC requires mono-sterylated oligonucleotides (steramers). Although not yet available commercially, it is show here that steramers can be obtained in >90% yield from commercial reagents on a DNA synthesizer. E-beacons incorporating steramers prepared by solid phase for SARS-COV-2 showed sensitive and sequence specific nucleic acid detection. For comparison, synthetic molecular beacons are used at ˜ 1 × 10⁻⁷ M; E-beacon signaling with the ultrabright Nluc/furimazine reaction are effective in the mid to low pM range. With so little E-beacon needed for each assay, a microgram-scale E-beacon preparation yields ample reagent for thousands of tests. Sensitivity is on par with the turn-off, two-component BRET-beacons. In the BRET beacon, an Nluc-oligo conjugate, generated by spontaneous chemical coupling, is hybridized to a second target-binding detector oligo. In this non-covalent assembly, BRET beacons report fully complementary target DNA by loss-of-fluorescence, rather than the gain-of-luminescence in the E-beacon. Specificity, the influence of base pair mismatches on fluorescence output with the BRET beacon, was not reported.

It is worthwhile to consider the potential challenges to translating E-beacons into a viable clinical diagnostic for pathogens like SARS-COV-2. Amplification of the patient specimen viral RNA, which can be in the attomolar range, would certainly be required to generate sufficient target nucleic acid to turn-on E-beacon signal. For point-of-care testing, isothermal amplification methods are favored which are completed in 30 to 60 minutes and carried out at ≤63° C. Preliminary experiments suggest that E-beacons are sufficiently thermostable to include directly in an RT-LAMP isothermal amplification reaction (FIG. 8). After 30 min. of heating at 63° C., template vs. non-template discrimination (S/N) was 10-fold, although the overall E-beacon bioluminescence was diminished. Alternative isothermal methods, such as NASBA and RPA, are carried out at lower temperatures, ˜40° C. and may prove more suitable. A second obstacle to the practical application of E-beacons involves finding a means to access the specimen, post-amplification, to allow addition of Nluc substrate. Accessing the sample must be done in a manner that avoids well-to-well contamination. For multi-well E-beacon assays, an adhesive plate seal that is amenable to both sample luminescence measurement and mechanical perforation (for Nluc substrate addition) would be required.

Unless otherwise defined, all technical and scientific terms used herein have the same meaning as commonly understood by one of ordinary skill in the art to which this invention belongs.

The embodiments illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that optional features, modification, improvement and variation of the embodiments herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

REFERENCES (All references are herein entirely incorporated by reference.): V. M. Corman, O. Landt, M. Kaiser, R. Molenkamp, A. Meijer, D. K. Chu, T. Bleicker, S. Brunink, J. Schneider, M. L. Schmidt, D. G. Mulders, B. L. Haagmans, B. van der Veer, S. van den Brink, L. Wijsman, G. Goderski, J. L. Romette, J. Ellis, M. Zambon, M. Peiris, H. Goossens, C. Reusken, M. P. Koopmans, C. Drosten, Euro Surveill 2020, 25; M. M. Arons, K. M. Hatfield, S. C. Reddy, A. Kimball, A. James, J. R. Jacobs, J. Taylor, K. Spicer, A. C. Bardossy, L. P. Oakley, S. Tanwar, J. W. Dyal, J. Harney, Z. Chisty, J. M. Bell, M. Methner, P. Paul, C. M. Carlson, H. P. Mclaughlin, N. Thornburg, S. Tong, A. Tamin, Y. Tao, A. Uehara, J. Harcourt, S. Clark, C. Brostrom-Smith, L. C. Page, M. Kay, J. Lewis, P. Montgomery, N. D. Stone, T. A. Clark, M. A. Honein, J. S. Duchin, J. A. Jernigan, H.-S. Public, C. King, C. C.-I. Team, N Engl J Med 2020, 382, 2081-2090; A. Hosseini, R. Pandey, E. Osman, A. Victorious, F. Li, T. Didar, L. Soleymani, ACS Sens 2020, 5, 3328-3345; D. B. Larremore, B. Wilder, E. Lester, S. Shehata, J. M. Burke, J. A. Hay, M. Tambe, M. J. Mina, R. Parker, Sci Adv 2020; Y. Zhang, G. Ren, J. Buss, A. J. Barry, G. C. Patton, N. A. Tanner, Biotechniques 2020; L. Zhou, A. R. Chandrasekaran, J. A. Punnoose, G. Bonenfant, S. Charles, O. Levchenko, P. Badu, C. Cavaliere, C. T. Pager, K. Halvorsen, Sci Adv 2020, 6; S. A. Marras, F. R. Kramer, S. Tyagi, Genet Anal 1999, 14, 151-156; S. Tyagi, F. R. Kramer, Nat Biotechno/1996, 14, 303-308; A. Moutsiopoulou, D. Broyles, E. Dikici, S. Daunert, S. K. Deo, Small 2019, 15, e1902248; M. P. Hall, J. Unch, B. F. Binkowski, M. P. Valley, B. L. Butler, M. G. Wood, P. Otto, K. Zimmerman, G. Vidugiris, T. Machleidt, M. B. Robers, H. A. Benink, C. T. Eggers, M. R. Slater, P. L. Meisenheimer, D. H. Klaubert, F. Fan, L. P. Encell, K. V. Wood, ACS Chem Biol 2012, 7, 1848-1857; H. Joda, A. Moutsiopoulou, G. Stone, S. Daunert, S. Deo, Analyst 2018, 143, 3374-3381; E. A. Hunt, S. K. Deo, Chem Commun (Camb) 2011, 47, 9393-9395; L. Biewenga, B. Rosier, M. Merkx, Biochem Soc Trans 2020, 48, 2643-2655; S. K. Elledge, X. X. Zhou, J. R. Byrnes, A. J. Martinko, I. Lui, K. Pance, S. A. Lim, J. E. Glasgow, A. A. Glasgow, K. Turcios, N. S. lyer, L. Torres, M. J. Peluso, T. J. Henrich, T. T. Wang, C. M. Tato, K. K. Leung, B. Greenhouse, J. A. Wells, Nat Biotechnol 2021; X. Zhang, Z. Xu, D. S. Moumin, D. A. Ciulla, T. S. Owen, R. A. Mancusi, J. L. Giner, C. Wang, B. P. Callahan, Bioconjug Chem 2019, 30, 2799-2804; D. A. Ciulla, A. G. Wagner, X. Liu, C. L. Cooper, M. T. Jorgensen, C. Wang, P. Goyal, N. K. Banavali, J. L. Pezzullo, J. L. Giner, B. P. Callahan, Chem Commun (Camb) 2019, 55, 1829-1832; G. Bonnet, S. Tyagi, A. Libchaber, F. R. Kramer, Proc Natl Acad Sci USA 1999, 96, 6171-6176; Oligonucleotide sterylation for the prototype E-beacons, Eb.1-3, was achieved by EDC coupling. After butanol extraction and RP-HPLC purification, the final isolated yield of the sterylated oligo varied between 30-40%. The efficiency of oligonucleotide sterylation was improved significantly by switching to solid phase methods with a DNA synthesizer, vide infra. We compared hybridization driven unquenching of with nuclease driven unquenching. Digestion of the hairpin component with DNASE-1 produced similar bioluminescence enhancement compared hybridization driven unquenching (Supporting FIG. 4); J. H. Zhang, T. D. Chung, K. R. Oldenburg, J Biomol Screen 1999, 4, 67-73.; See e.g., the website at covdb.stanford.edu/page/mutation-viewer; W. Engelen, K. M. van de Wiel, L. H. H. Meijer, B. Saha, M. Merkx, Chem Commun (Camb) 2017, 53, 2862-2865; R. Arnaout, R. A. Lee, G. R. Lee, C. Callahan, A. Cheng, C. F. Yen, K. P. Smith, R. Arora, J. E. Kirby, Clin Infect Dis 2021, 73, e3042-e3046; S. Sherrill-Mix, Y. Hwang, A. M. Roche, A. Glascock, S. R. Weiss, Y. Li, L. Haddad, P. Deraska, C. Monahan, A. Kromer, J. Graham-Wooten, L. J. Taylor, B. S. Abella, A. Ganguly, R. G. Collman, G. D. Van Duyne, F. D. Bushman, Genome Biol 2021, 22, 169; Y. Zhang, N. A. Tanner, Biotechniques 2021, 70, 167-174.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

What is claimed is:

1. A composition, comprising a conjugate reaction product of:
a signal emitting polypeptide or functional fragment thereof having a glycine amino acid residue located at the final residue position of the light emitting polypeptide;
a linker characterized as a fused sterol or stanol ring system, having a nucleophilic group at a 3-position of an A-ring of a fused sterol or stanol ring system with beta or alpha stereochemistry; and
at least one oligonucleotide having a 5′ end and a 3′ end,
wherein the composition is characterized by one of the 3′ end or the 5′ end attached to the linker and one of the 3′ end or the 5′ end attached to a quencher, and wherein the linker is covalently linked to the glycine amino acid residue.

2. The composition of claim 1, wherein the oligonucleotide is characterized as a single-stranded nucleic acid.

3. The composition of claim 1, wherein the oligonucleotide is DNA or RNA.

4. The composition of claim 1, wherein the oligonucleotide comprises a hair-pin loop.

5. The composition of claim 1, wherein the reaction product has a first conformation in an absence of a predetermined target strand and a second conformation in a presence of a predetermined target strand, if any.

6. The composition of claim 5, further comprising an indicator under conditions sufficient to form a signal.

7. The composition of claim 6, wherein the indicator is at least one selected from the group consisting of furimazine, coelenterazine and a luciferin.

8. The composition of claim 1, wherein the reaction product further comprises a predetermined target strand hybridized to the oligonucleotide.

9. The composition of claim 1, wherein the signal emitting peptide is a luciferase enzyme.

10. The composition of claim 1, wherein the signal emitting peptide is an enzyme or functional fragment thereof.

11. The composition of claim 1, wherein in the quencher is an agent capable of decreasing the radiative output from the signal emitting peptide.

12. The composition according to claim 1, wherein the oligonucleotide has a first segment and a second segment and a third segment, wherein the first and second segment are complementary and form a self-annealed stem structure and the third segment forms a loop that is preselected to hybridize to a preselected analyte of interest.

13. The composition according to claim 1, wherein the oligonucleotide is preselected to hybridize to a preselected analyte of interest.

14. The composition of claim 1, wherein the oligonucleotide is preselected to hybridize to viral DNA or viral RNA.

15. The composition according to claim 1, wherein the oligonucleotide has a first confirmation characterized as closed and, when contacted with a preselected nucleic acid, forms a second conformation characterized as signal emitting.

16. A composition, comprising: a peptide reporter molecule, and a hairpin-forming mono-sterylated oligonucleotide comprising a quencher.

17. The composition of claim 16, wherein the peptide reporter molecule is a light emitting polypeptide or light emitting enzyme.

18. The composition of claim 17, wherein the light emitting enzyme is a luciferase enzyme.

19. A method of making a composition, comprising:
contacting a light emitting polypeptide or functional fragment thereof having a glycine amino acid residue located at the final residue position of the light emitting polypeptide with a linker characterized as a fused sterol or stanol ring system having a nucleophilic group at a 3-position of an A-ring of a fused sterol or stanol ring system with beta or alpha stereochemistry, and an oligonucleotide having a 5′ end and a 3′ end or the 5′ end or the 3′ end attached to a quencher.

20. The method of claim 19, wherein the light emitting polypeptide or functional fragment thereof is an enzyme.

21. A method of determining a presence of an analyte of interest, comprising:
contacting a composition comprising a light emitting polypeptide or functional fragment thereof, a linker characterized as a fused sterol or stanol ring system, and an oligonucleotide having a 5′ end attached to the linker and a 3′ end attached to a quencher with an analyte of interest under conditions sufficient to separate the light emitting polypeptide or functional fragment thereof from the quencher to generate a signal.

22. The method of claim 21, wherein the oligonucleotide is characterized as a single-stranded nucleic acid.

23. The method of claim 22, wherein the oligonucleotide is DNA or RNA.

24. The method of claim 21, wherein the oligonucleotide comprises a hair-pin loop.

25. The method according to claim 22, wherein the oligonucleotide has a first segment, a second segment, and a third segment, wherein the first segment and second segment form a self-annealed stem structure and the third segment forms a loop that is preselected to hybridize to a preselected analyte of interest.

26. The method of claim 21, wherein the composition further comprising an indicator under conditions sufficient to form a signal.

27. The method of claim 26, wherein the indicator is at least one selected from the group consisting of furimazine, coelenterazine and a luciferin.

28. A method of reconfiguring a protein-nucleic acid fusion molecule, comprising:
contacting a protein-nucleic acid fusion molecule, having a first conformation, with an oligonucleotide of interest to form a protein-nucleic acid fusion molecule complex having a second conformation, wherein the second conformation is characterized as on; and
contacting the second conformation with an indicator under conditions sufficient to form a signal.

29. The method of claim 28, wherein the signal is predetermined to show a presence or absence of oligonucleotide of interest.

30. The composition of claim 1, wherein the quencher is a dark fluorescence quencher.


SEQUENCE LISTING (13 sequences)

SEQ ID NO: 1 - DNA, 25 bp, synthetic construct
  source 1..25: mol_type = other DNA
cgctcccaaaaaaaaaaaccgagcg

SEQ ID NO: 2 - DNA, 25 bp, synthetic construct
  source 1..25: mol_type = other DNA
cgctctggtgttgaaggtttgagcg

SEQ ID NO: 3 - DNA, 25 bp, synthetic construct
  source 1..25: mol_type = other DNA
cgctctggtgttaaaggtttgagcg

SEQ ID NO: 4 - DNA, 15 bp, synthetic construct
  source 1..15: mol_type = other DNA
ggtttttttttttgg

SEQ ID NO: 5 - DNA, 15 bp, synthetic construct
  source 1..15: mol_type = other DNA
ctggtcttcgggcta

SEQ ID NO: 6 - DNA, 15 bp, synthetic construct
  source 1..15: mol_type = other DNA
ggttttgttttttgg

SEQ ID NO: 7 - DNA, 15 bp, synthetic construct
  source 1..15: mol_type = other DNA
ggttttggtttttgg

SEQ ID NO: 8 - DNA, 15 bp, synthetic construct
  source 1..15: mol_type = other DNA
ggttttgggttttgg

SEQ ID NO: 9 - DNA, 15 bp, synthetic construct
  source 1..15: mol_type = other DNA
gggtttttttttggg

SEQ ID NO: 10 - DNA, 15 bp, synthetic construct
  source 1..15: mol_type = other DNA
ggttttcccttttgg

SEQ ID NO: 11 - DNA, 15 bp, synthetic construct
  source 1..15: mol_type = other DNA
aaaccttcaacacca

SEQ ID NO: 12 - DNA, 15 bp, synthetic construct
  source 1..15: mol_type = other DNA
aaacctttaacacca

SEQ ID NO: 13 - DNA, 15 bp, synthetic construct
  source 1..15: mol_type = other DNA
aaaccttgaacacca